(19) United States
(12) Patent Application Publication
(10) Pub. No.: US 2013/0192647 A1
(43) Pub. Date: Aug. 1, 2013

(54) ENDOSCOPIC COMPONENT CLEANING SYSTEM AND METHOD

(71) Applicants: Jeffrey William Ledel, Shakopee, MN (US); Daniel L. Cummings, Wayzata, MN (US); Geoffrey D. Tuttle, Minnetonka, MN (US)

(72) Inventors: Jeffrey William Ledel, Shakopee, MN (US); Daniel L. Cummings, Wayzata, MN (US); Geoffrey D. Tuttle, Minnetonka, MN (US)

(21) Appl. No.: 13/745,262

(22) Filed: Jan. 18, 2013

Related U.S. Application Data
(63) Continuation-in-part of application No. 13/552,107, filed on Jul. 18, 2012.
(60) Provisional application No. 61/509,012, filed on Jul. 18, 2011.

(51) Int. Cl. A61B 1/12

(52) U.S. Cl. CPC A61B 1/123
USPC 134/22.1; 134/166 R

(57) ABSTRACT

Cleaning systems and methods include use of a container or holding apparatus to receive one or more components used in a medical procedure (e.g., a walled container or a non-walled container including a plurality of holding structures) and a manifold apparatus. For example, the manifold apparatus may include an inlet connection connectable to a reprocessor fitting of a reprocessing apparatus to receive a fluid therefrom (e.g., a cleaning solution, a disinfecting solution, and a rinsing solution) and one or more outlets configured to provide the at least one fluid into or through one or more components received by the container. For example, the one or more outlets may include an outlet connection connectable to a component fitting of at least one of the components.

### CROSS-REFERENCE TO RELATED APPLICATIONS

This application is a continuation-in-part of U.S. application Ser. No. 13/552,107 filed 18 Jul. 2012, entitled “ENDOSCOPIC COMPONENT CLEANING SYSTEM AND METHOD” which claims the benefit under 35 U.S.C. §119(e) of U.S. Provisional Application Ser. No. 61/509,012 filed 18 Jul. 2011, entitled “GI ENDOSCOPIC IRRIGATION CLEANING SYSTEM,” which are all incorporated herein by reference in their entirety.

### BACKGROUND

The present disclosure pertains generally to systems and methods to clean components used in medical procedures (e.g., such as water bottles, water bottle caps, irrigation tubing, etc. used in endoscopic procedures).

Many invasive medical procedures that previously required major surgery are now performed using endoscopic instruments. Such instruments may provide an internal view of particular body parts, organs, or passages without requiring invasive surgery. Generally, an endoscopic instrument may include one or more channels through which miniaturized, flexible instruments can be inserted and advanced. The endoscope may include an elongated flexible cable equipped at one end with an eyepiece or other viewing apparatus and at the other end with an optical head (e.g., an illuminating apparatus often involving optical bundles may be used to illuminate a desired area).

The cable of an endoscope may also provide a flow passage for the delivery of fluid (e.g., liquid or gas) for irrigation or other purposes. For example, the flow passage may be used to provide a flow of sterile water across the optic head to prevent the buildup of materials (e.g., surgical debris and body fluids) on the optic head.

Endoscopes are generally not disposable single-use devices, e.g., they are used repeatedly and on more than one patient. For an endoscope to be repeatedly used in this way, it must be meticulously reprocessed after each use in order to prevent endoscopically transmitted bacterial infections. In other words, after use of an endoscope, it is generally cleaned or reprocessed. For example, reprocessing an endoscope may refer to performing a cleaning and disinfecting process on an endoscope. Such processes may include, for example, cleaning an endoscope with a liquid detergent (e.g., a cleaning step), disinfecting with a liquid disinfectant the endoscope that has been cleaned with detergent (e.g., a disinfecting step), and rinsing the endoscope that has been disinfected with disinfectant (e.g., a rinsing step). To this end, various types of endoscope reprocessors for the automated reprocessing of endoscopes have been developed for commercial use. Generally, such automated endoscopic reprocessors (AERs) include a reprocessing chamber (e.g., a basin) into which the endoscope can be placed. Within the chamber, a connection apparatus is generally available to fluidly connect to one or more portions of the endoscope (e.g., an AER fitting compatible with a fitting of the endoscope) so as to be able to provide fluid for flushing out the endoscope (e.g., providing fluid therethrough or provide various fluids to be circulated therein for cleaning the endoscope). Such AERs may also include fluid ports open into the reprocessing chamber to provide fluid, such as, for example, to spray the outer portions of the endoscope or bathe the endoscope.

In many exemplary designs, there may be a great expense associated with the delivery of sterile water in an endoscopy system. A known practice has been to use a water bottle with a cap having a tube running therethrough. The tube typically has a fitting at the end distal to the bottle to allow for connection to, for example, a port of the endoscopic system, such as an air/water connection port. Further, for example, the tube connecting the water bottle to the endoscope may be formed of an inner tube and an outer tube (e.g., the inner tube may extend into the water bottle and the outer tube may be connected to the cap of the water bottle). For example, air may be delivered through the area between the inner tube and the outer tube so as to pressurize the interior of the water bottle and force water to flow through the tube and into the endoscope at a desired rate.

Such known water bottle configuration may present several problems, e.g., such as cost and sterilization. For example, failure to properly clean and disinfect endoscopic equipment after each examination can compromise patient safety. For example, every channel of the endoscope may need to be reprocessed each time the endoscope is used, even if the channel was not utilized during the preceding patient procedure. Further, for example, every channel may need to go through each the entire reprocessing procedure (e.g., cleaning, disinfection, rinsing, alcohol/air drying). This creates a considerable expense to the hospital, including a considerable labor expense associated with the disinfection of the water bottle set or configuration. It is not cost effective to simply dispose of a water bottle set after a single use because of the expense associated with the water bottle/cap/tubing configurations. However, disposable water bottle components are currently available.

### SUMMARY

The present disclosure presents a solution to these and other problems associated with water bottle configurations used in endoscopy systems, as well as a solution that may be used with the cleaning of components used in other medical procedures.

One exemplary cleaning system of the present disclosure (e.g., including a walled container or a non-walled container including a plurality of holding structures) may be for use with an automated endoscopic reprocessing apparatus for cleaning one or more components used in an endoscopic medical procedure. The automated endoscopic reprocessing apparatus may include a reprocessor chamber and a reprocessor fitting configured to provide at least one fluid (e.g., at least one of a cleaning solution, a disinfecting solution, and a rinsing solution). Further, the one or more components may include at least one of one or more water bottles, one or more water bottle caps, and one or more irrigation tubes used in an endoscopic procedure. For example, each water bottle cap may include at least one tube therethrough terminating at a tube fitting connectable to an endoscopic system used in an endoscopic procedure and each irrigation tube may extend from a first end to a second end with at least one of the first and second ends terminating at a tube fitting connectable to an endoscopic system used in an endoscopic procedure.

In one or more embodiments, the cleaning system may include a container to receive and hold the one or more components and a manifold apparatus. For example, the container may include a walled container that includes at least a bottom and one or more side walls, wherein at least portions of the bottom and one or more side walls comprise a network of material providing a plurality of openings to allow fluids to pass therethrough to contact the one or more components. Further, for example, the container may include a non-walled container that includes a plurality of holding structures, wherein each of the plurality of holding structures is configured to receive at least one component of the one or more components such that fluid is allowed to contact the one or more components (e.g., the non-walled container may be configured as at least one rack apparatus including a bottom structure relative to which one or more of the plurality of holding structures are arranged to receive the one or more components).

In one or more embodiments, the exemplary cleaning system may include a container to receive the one or more components (e.g., a non-collapsible walled container). The container may include at least a bottom and one or more side walls, wherein at least portions of the bottom and one or more side walls may include a network of material providing a plurality of openings to allow fluids to pass therethrough to contact the one or more components (e.g., a wire mesh, a wire grid, a metal material defining a plurality of openings, plastic or molded plastic material defining a plurality of openings, etc.). The cleaning system may further include a manifold apparatus configured to be coupled to the container. The manifold apparatus may include an inlet connection connectable to the reprocessor fitting of the automated endoscopic reprocessing apparatus to receive the at least one fluid therefrom and one or more outlets configured to provide the at least one fluid into or through the one or more components. The one or more outlets may include at least one outlet connection connectable to at least one tube fitting of at least one water bottle cap or at least one irrigation tube.

In one or more embodiments of the exemplary cleaning system, the manifold apparatus may further include one or more manifold tube portions fluidly connected in the path from the inlet connection to the one or more outlets (e.g., at least one of the one or more manifold tube portions may include an outlet terminating a tube portion extending and pointing away from the bottom of the container when the manifold apparatus is coupled to the container such that an open end of the water bottle is positionable onto the tube portion).

Further, in one or more embodiments of the exemplary cleaning system, the system may further include one or more attachment elements to secure the one or more irrigation tubes to the network of material of the container in a manner allowing the at least one irrigation tube to gravity drain.

Further, in one or more embodiments of the exemplary cleaning system, the one or more components may include at least one water bottle, at least one water bottle cap, and at least one irrigation tube used in an endoscopic procedure. The one or more outlets of the manifold apparatus may include at least one outlet connection connectable to a tube fitting of the at least one water bottle cap and at least one outlet connection connectable to a tube fitting of the at least one irrigation tube. Further, the manifold apparatus may include one or more manifold tube portions fluidly connected in the path from the inlet connection to the one or more outlets; at least one of the one or more manifold tube portions may include an outlet terminating a tube portion extending and pointing away from the bottom of the container when the manifold apparatus is coupled to the container such that an open end of the at least one water bottle is positionable onto the tube portion.

Another exemplary cleaning system described in the present disclosure may be used with a reprocessing apparatus for cleaning one or more components used in a medical procedure. The cleaning system may include a container to receive the one or more components. The container may include at least a bottom and one or more side walls. At least portions of the bottom and one or more side walls may include a network of material providing a plurality of openings to allow fluid to pass therethrough to contact the one or more components. The cleaning system may further include a manifold apparatus configured to be coupled to the container. The manifold apparatus may include an inlet connection connectable to a reprocessor fitting of the reprocessing apparatus to receive at least one fluid therefrom (e.g., a cleaning solution, a disinfecting solution, a rinsing solution, etc.) and one or more outlets configured to provide the at least one fluid into or through one or more components received by the container. The one or more outlets may include at least one outlet connection connectable to a component fitting of at least one of the one or more components.

An exemplary method of cleaning one or more components used in a medical procedure is also described herein. The cleaning method may include providing a reprocessing apparatus that includes a reprocessing chamber and a reprocessor fitting through which at least one fluid is provided, wherein the at least one fluid comprises at least one of a cleaning solution, a disinfecting solution, and a rinsing solution, and providing a cleaning system. The cleaning system may include a container to receive and hold the one or more components and a manifold apparatus. The manifold apparatus may include an inlet connection connectable to the reprocessor fitting of the reprocessing apparatus to receive the at least one fluid therefrom and one or more outlets configured to provide the at least one fluid into or through the one or more components (e.g., wherein the one or more outlets may include at least one outlet connection connectable to a component fitting of at least one of the one or more components). Further, the method may include receiving at least one of the one or more components by the container (e.g., wherein receiving that at least one component may include connecting the at least one outlet connection to the component fitting of the at least one component) and positioning the container into the reprocessing chamber (e.g., wherein positioning the container into the reprocessing chamber may include connecting the inlet connection to the reprocessor fitting of the reprocessing apparatus). The at least one fluid may be provided into or through at least one of the one or more components positioned into the reprocessing chamber.

In one or more embodiment, the one or more components may include at least one of a water bottle, a water bottle cap, and an irrigation tube used in an endoscopic procedure, and further wherein the inlet connection of the manifold apparatus may be connectable to a reprocessor fitting of an automated endoscopic reprocessing apparatus from which it receives the at least one fluid.

In one or more embodiments, the container may include a walled container that includes at least a bottom and one or more side walls, wherein at least portions of the bottom and one or more side walls may include a network of material providing a plurality of openings to allow fluids to pass therethrough to contact the one or more components. In other embodiments, the container may include a non-walled container that include a plurality of holding structures, wherein each of the plurality of holding structures may be configured to receive at least one component of the one or more components such that fluid is allowed to contact the one or more components (e.g., the non-walled container may be configured as at least one rack apparatus including a bottom structure relative to which one or more of the plurality of holding structures are arranged to receive the one or more components).

Further, in one or more embodiments, the manifold apparatus may include one or more manifold tube portions fluidly connected in the path from the inlet connection to the one or more outlets, wherein at least one of the one or more manifold tube portions may include an outlet insertable into an interior volume of at least one of the one or more components (e.g., the at least one manifold tube portion may include an outlet terminating a tube portion extending in a direction such that an open end of the water bottle is positionable onto the tube portion).

Further, in one or more embodiments, the one or more components may include at least one of a water bottle cap used in an endoscopic procedure, the water bottle cap may include at least one tube therethrough terminating at a tube fitting connectable to an endoscopic system used in an endoscopic procedure, and the at least one outlet connection of the one or more outlets of the manifold apparatus may be connectable to the tube fitting of the at least one tube extending through the water bottle cap.

Further, in one or more embodiments, the one or more components may include at least one irrigation tube used in an endoscopic procedure, the irrigation tube may extend from a first end to a second end with at least one of the first and second ends terminating at a tube fitting connectable to an endoscopic system used in an endoscopic procedure, and the at least one outlet connection of the one or more outlets of the manifold apparatus may be connectable to the tube fitting of the at least one irrigation tube.

Another cleaning system for use with a reprocessing apparatus for cleaning one or more components used in a medical procedure may include at least one rack apparatus including a bottom structure relative to which one or more of a plurality of holding structures are arranged to receive the one or more components and a manifold apparatus. The manifold apparatus may include an inlet connection connectable to a reprocessor fitting of the reprocessing apparatus to receive at least one fluid therefrom (e.g., wherein the at least one fluid may include at least one of a cleaning solution, a disinfecting solution, and a rinsing solution) and one or more outlets configured to provide the at least one fluid into or through one or more components received by the at least one rack apparatus (e.g., wherein the one or more outlets may include at least one outlet connection connectable to a component fitting of at least one of the one or more components).

In one or more embodiments, the one or more components may include at least one of a water bottle, a water bottle cap, and an irrigation tube used in an endoscopic procedure, and further wherein the inlet connection of the manifold apparatus may be connectable to a reprocessor fitting of an automated endoscopic reprocessing apparatus from which it receives the at least one fluid.

In one or more embodiments, the manifold apparatus may include one or more manifold tube portions fluidly connected in the path from the inlet connection to the one or more outlets, wherein at least one of the one or more manifold tube portions may include an outlet insertable into an interior volume of at least one of the one or more components.

Further, in one or more embodiments, the one or more components may include at least one water bottle used in an endoscopic procedure, wherein the at least one manifold tube portion may include an outlet terminating a tube portion extending in a direction such that an open end of the water bottle is positionable onto the tube portion.

Further, in one or more embodiments, the one or more components may include at least one of a water bottle cap used in an endoscopic procedure, the water bottle cap may include at least one tube therethrough terminating at a tube fitting connectable to an endoscopic system used in an endoscopic procedure, and the at least one outlet connection of the one or more outlets of the manifold apparatus may be connectable to the tube fitting of the at least one tube extending through the water bottle cap.

Further, in one or more embodiments, the one or more components may include at least one irrigation tube used in an endoscopic procedure, the irrigation tube may extend from a first end to a second end with at least one of the first and second ends terminating at a tube fitting connectable to an endoscopic system used in an endoscopic procedure, and the at least one outlet connection of the one or more outlets of the manifold apparatus may be connectable to the tube fitting of the at least one irrigation tube (e.g., at least one holding structure of the plurality of holding structures may include one or more retaining elements used to secure the at least one irrigation tube in a manner allowing the at least one irrigation tube to gravity drain).

Further, in one or more embodiments, at least one holding structure of the plurality of holding structures may include one or more retaining elements insertable into at least a portion of at least one component of the one or more components to maintain the at least one component in a position when fluid is allowed to contact the at least one component, may include one or more retaining elements that engage at least a surface portion of at least one component of the one or more components to maintain the at least one component in a position when fluid is allowed to contact the at least one component, and/or may include one or more retaining elements into which at least a portion of at least one component of the one or more components is insertable to maintain the at least one component in a position when fluid is allowed to contact the at least one component.

Further, in one or more embodiments, each of the plurality of holding structures may be configured to receive at least one component of the one or more components and maintain the at least one component in a position when fluid is allowed to contact the one or more components. Still further, in one or more embodiments, the manifold apparatus may be coupled to the at least one rack apparatus.

In one or more embodiments of the cleaning systems or methods, the one or more components may include at least one of a water bottle, a water bottle cap, and an irrigation tube used in an endoscopic procedure, and further the inlet connection of the manifold apparatus may be connectable to a reprocessor fitting of an automated endoscopic reprocessing apparatus from which it receives the at least one fluid.

Further, in one or more embodiments of the cleaning systems or methods, the manifold apparatus may include one or more manifold tube portions fluidly connected in the path from the inlet connection to the one or more outlets. At least one of the one or more manifold tube portions may include an outlet insertable into an interior volume of at least one of the one or more components.

Further, in one or more embodiments of the cleaning systems or methods, the one or more components may include at least one water bottle used in an endoscopic procedure and the at least one manifold tube portion may include an outlet terminating a tube portion extending and pointing away from the bottom of the container when the manifold apparatus is coupled to the container such that an open end of the water bottle is positionable onto the tube portion (e.g., the outlet terminating the tube portion extending and pointing away from the bottom of the container may include a spray element).

Still further, in one or more embodiments of the cleaning systems or methods, the one or more components may include at least one of a water bottle cap used in an endoscopic procedure (e.g., the water bottle cap may include at least one tube therethrough terminating at a tube fitting connectable to an endoscopic system used in an endoscopic procedure) and the at least one outlet connection of the one or more outlets of the manifold apparatus may be connectable to the tube fitting of the at least one tube extending through the water bottle cap.

Still further, in one or more embodiments of the cleaning systems or methods, the one or more components may include at least one irrigation tube used in an endoscopic procedure (e.g., the irrigation tube may extend from a first end to a second end with at least one of the first and second ends terminating at a tube fitting connectable to an endoscopic system used in an endoscopic procedure) and further the at least one outlet connection of the one or more outlets of the manifold apparatus may be connectable to the tube fitting of the at least one irrigation tube.

Another exemplary cleaning system for use with a reprocessing apparatus for cleaning one or more components used in a medical procedure may include a non-walled container to receive the one or more components (e.g., wherein the non-walled container may include a plurality of holding structures, wherein each of the plurality of holding structures may be configured to receive at least one component of the one or more components such fluid is allowed to contact the one or more components one, and further wherein each of the plurality of holding structures may maintain the at least one component in a position when fluid is allowed to contact the one or more components). Further, the cleaning system may include a manifold apparatus (e.g., that may be configured to be coupled to the non-walled container). The manifold apparatus may include an inlet connection connectable to a reprocessor fitting of the reprocessing apparatus to receive at least one fluid therefrom (e.g., the at least one fluid may include at least one of a cleaning solution, a disinfecting solution, and a rinsing solution) and one or more outlets configured to provide the at least one fluid into or through one or more components received in the container (e.g., the one or more outlets may include at least one outlet connection connectable to a component fitting of at least one of the one or more components).

Further, another exemplary method of cleaning one or more components used in a medical procedure may include providing a reprocessing apparatus (e.g., that includes a reprocessing chamber and a reprocessor fitting through which at least one fluid is provided, wherein the at least one fluid comprises at least one of a cleaning solution, a disinfecting solution, and a rinsing solution) and providing a cleaning system. For example, the cleaning system may include a non-walled container to receive the one or more components (e.g., wherein the non-walled container may include a plurality of holding structures, wherein each of the plurality of holding structures may be configured to receive at least one component of the one or more components such fluid is allowed to contact the one or more components one, and further wherein each of the plurality of holding structures may maintain the at least one component in a position when fluid is allowed to contact the one or more components) and a manifold apparatus (e.g., that may be configured to be coupled to the non-walled container). The manifold apparatus may include an inlet connection connectable to the reprocessor fitting of the reprocessing apparatus to receive the at least one fluid therefrom and one or more outlets configured to provide the at least one fluid into or through the one or more components (e.g., the one or more outlets may include at least one outlet connection connectable to a component fitting of at least one of the one or more components). Further, the method may include positioning at least one component of the one or more components relative to at least one holding structure of the plurality of holding structures (e.g., positioning the at least one component may include connecting the at least one outlet connection to the component fitting of the at least one component) and positioning the non-walled container into the reprocessing chamber (e.g., positioning the non-walled container into the reprocessing chamber may include connecting the inlet connection to the reprocessor fitting of the reprocessing apparatus). Still further, the method may include providing the at least one fluid into or through at least one of the one or more components.

In one or more embodiments, the non-walled container may be configured as at least one rack apparatus including a bottom structure relative to which one or more of the plurality of holding structures are arranged to receive the one or more components.

In one or more embodiments of the cleaning system or method (e.g., one using a non-walled container), the one or more components may include at least one of a water bottle, a water bottle cap, and an irrigation tube used in an endoscopic procedure, and further, the inlet connection of the manifold apparatus may be connectable to a reprocessor fitting of an automated endoscopic reprocessing apparatus from which it receives the at least one fluid.

In one or more embodiments of the cleaning system or method (e.g., one using a non-walled container), the manifold apparatus may include one or more manifold tube portions fluidly connected in the path from the inlet connection to the one or more outlets, wherein at least one of the one or more manifold tube portions may include an outlet insertable into an interior volume of at least one of the one or more components.

Still further, in one or more embodiments of the cleaning system or method (e.g., one using a non-walled container), the one or more components may include at least one water bottle used in an endoscopic procedure, wherein the at least one manifold tube portion may include an outlet terminating a tube portion extending in a direction such that an open end of the water bottle is positionable onto the tube portion (e.g., the outlet terminating the tube portion may include a spray element).

Further, in one or more embodiments of the cleaning system or method (e.g., one using a non-walled container), the one or more components may include at least one of a water bottle cap used in an endoscopic procedure (e.g., a water bottle cap that may include at least one tube therethrough terminating at a tube fitting connectable to an endoscopic system used in an endoscopic procedure), wherein the at least one outlet connection of the one or more outlets of the manifold apparatus may be connectable to the tube fitting of the at least one tube extending through the water bottle cap.

Yet further, in one or more embodiments of the cleaning system or method (e.g., one using a non-walled container), the one or more components may include at least one irrigation tube used in an endoscopic procedure (e.g., the irrigation tube may extend from a first end to a second end with at least one of the first and second ends terminating at a tube fitting connectable to an endoscopic system used in an endoscopic procedure), wherein the at least one outlet connection of the one or more outlets of the manifold apparatus may be connectable to the tube fitting of the at least one irrigation tube. Further, for example, at least one holding structure of the plurality of holding structures may include one or more retaining elements used to secure the at least one irrigation tube in a manner allowing the at least one irrigation tube to gravity drain.

Yet further, in one or more embodiments of the cleaning system or method (e.g., one using a non-walled container), at least one holding structure of the plurality of holding structures may include one or more retaining elements insertable into at least a portion of at least one component of the one or more components to maintain the at least one component in a position when fluid is allowed to contact the at least one component, one or more retaining elements that engage at least a surface portion of at least one component of the one or more components to maintain the at least one component in a position when fluid is allowed to contact the at least one component, and/or at least one of one or more retaining elements into which at least a portion of at least one component of the one or more components is insertable to maintain the at least one component in a position when fluid is allowed to contact the at least one component.

In addition, in one or more embodiments of the cleaning system or method (e.g., one using a non-walled container), the one or more components may include at least a water bottle and a water bottle cap used in an endoscopic procedure, wherein the inlet connection of the manifold apparatus is connectable to a reprocessor fitting of an automated endoscopic reprocessing apparatus from which it receives the at least one fluid, and further wherein the non-walled container may include a plurality of holding structures sized to be received in a reprocessing chamber of the endoscopic reprocessing apparatus.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a general diagram for use in describing one or more exemplary embodiments of a cleaning system usable in an automated reprocessor.

FIG. 2 shows a perspective view of one embodiment of a cleaning system with endoscopic components to be cleaned.

FIG. 3 shows a perspective view of the cleaning system of FIG. 2 in an empty state.

FIG. 4 shows a perspective view of the cleaning system of FIG. 2 with a lid thereof closed.

FIG. 5 shows a perspective view of an exemplary water bottle cap used in an endoscopic procedure.

FIG. 6 shows a perspective view of an exemplary water bottle used in an endoscopic procedure.

FIG. 7 shows a perspective view of exemplary irrigation tubing used in an endoscopic procedure.

FIG. 8 shows an exploded perspective view of another embodiment of a cleaning system including the endoscopic components to be cleaned, and with a lid of the system.

FIG. 9 shows a perspective view of the cleaning system of FIG. 8 with endoscopic components to be cleaned, and with a lid of the system removed.

FIG. 10 shows a top view of the cleaning system of FIG. 8 including the endoscopic components to be cleaned.

FIG. 11 shows a top view of the cleaning system of FIG. 8 with endoscopic components removed.

FIG. 12 shows a side view of the cleaning system of FIG. 8 with endoscopic components removed.

FIG. 13 shows a cross-section view of the cleaning system of FIG. 8 taken along line A-A as shown in FIG. 10.

FIG. 14 shows a cross-section view of the cleaning system of FIG. 8 taken along line B-B as shown in FIG. 13.

FIG. 15 shows a top view of an exemplary manifold apparatus used in the cleaning system of FIG. 8 with endoscopic components connected for cleaning.

FIG. 16 shows a side view of the exemplary manifold apparatus with endoscopic components connected for cleaning as shown in FIG. 15.

FIG. 17 shows a side perspective view of the exemplary manifold apparatus shown in FIGS. 15-16 used in the cleaning system of FIG. 8 with endoscopic components removed.

FIG. 18 shows a top view of the exemplary manifold apparatus with endoscopic components removed as shown in FIG. 17.

FIG. 19 shows an exploded perspective view of another embodiment of a cleaning system including a non-walled container, manifold apparatus, and the endoscopic components to be cleaned.

FIG. 20 shows a perspective view of the cleaning system of FIG. 19 with endoscopic components in position to be cleaned.

FIG. 21 shows a top view of the cleaning system of FIG. 19 including the endoscopic components to be cleaned.

FIG. 22 shows a top view of the cleaning system of FIG. 19 with endoscopic components removed.

FIG. 23 shows a side view of the cleaning system of FIG. 19 with endoscopic components removed.

FIG. 24 shows a cross-section view of the cleaning system of FIG. 19 taken along line A-A as shown in FIG. 21.

FIG. 25 shows a cross-section view of the cleaning system of FIG. 19 taken along line B-B as shown in FIG. 21.

FIG. 26 shows a side view of at least a portion of the exemplary manifold apparatus with endoscopic components connected for cleaning.

FIG. 27 shows a side perspective view of at least a portion of the exemplary manifold apparatus used in the cleaning system of FIG. 19 with endoscopic components removed.

### DETAILED DESCRIPTION OF EXEMPLARY EMBODIMENTS

In the following detailed description of illustrative embodiments, reference is made to the accompanying figures of the drawing which form a part hereof, and in which are shown, by way of illustration, specific embodiments which may be practiced. It is to be understood that other embodiments may be utilized and structural changes may be made without departing from (e.g., still falling within) the scope of the disclosure presented hereby.

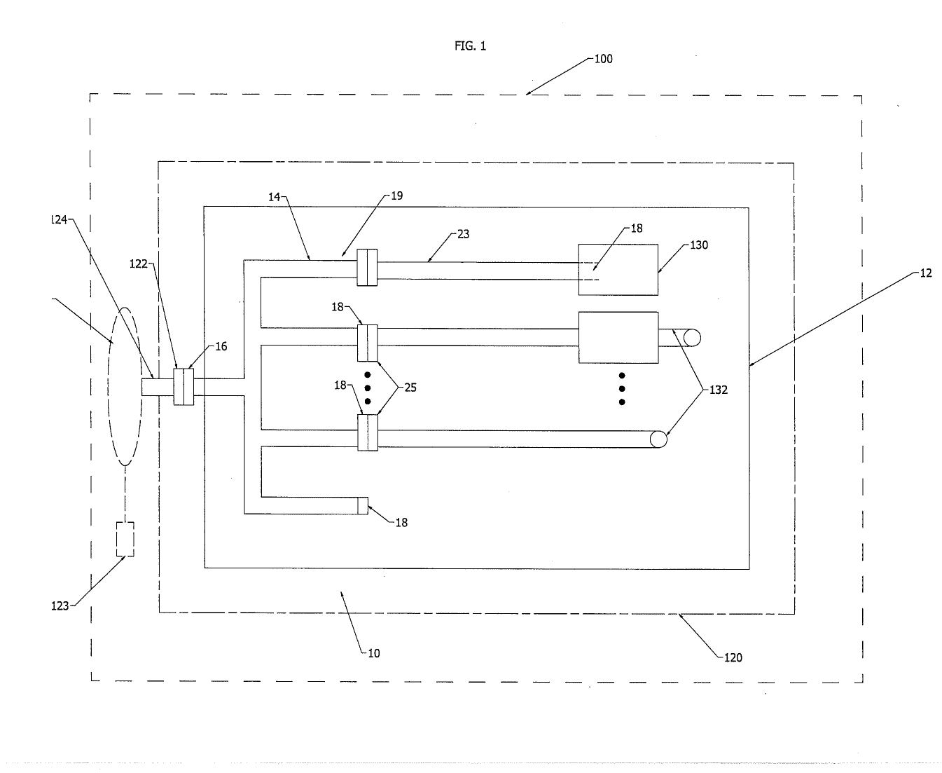

Exemplary methods, apparatus, and systems shall be described with reference to FIGS. 1-27. It will be apparent to one skilled in the art that elements or processes from one embodiment may be used in combination with elements or processes of the other embodiments, and that the possible embodiments of such methods and systems using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

FIG. 1 is a general diagram for use in describing one or more exemplary embodiments of a cleaning system 10 for use with a reprocessing apparatus 100. The cleaning system 10 may be used with any suitable reprocessing apparatus (e.g., an automated endoscopic reprocessing system) for use in cleaning one or more components used in a medical procedure (e.g., an endoscopic medical procedure). For example, such suitable reprocessing apparatus may include any known and/or available reprocessor such as, ASP reprocessors (Ethicon—a division of Johnson & Johnson, Irvine Calif.), reprocessors available under the trade designation Medivators-DSD, Advantage, Advantage Plus, etc. (Cantel Company, Minneapolis Minn.), Custom Ultrasonics System 83 Plus 2 and System 83 Plus 9 (Custom Ultrasonics, Ivyland, Pa.), Olympus America OER Pro reprocessors, etc. (Olympus America, Canterville, Pa.), Steris Corp. reprocessors available under the trade designation Amsco V pro, Hamo EndoClean PTX, System 1, and Reliance System (Steric Corp., Mentor Ohio). In one or more embodiment, the reprocessing apparatus 100 is an automated endoscopic reprocessor suitable for reprocessing endoscopes.

In one or more embodiments, the reprocessing apparatus 100 may include a reprocessing chamber 120. For example, such reprocessing chambers are utilized in reprocessors to position one or more medical devices to be reprocessed. Further, for example, with respect to an automated endoscopic reprocessor, such a reprocessing chamber 120 may be a basin into which one or more medical devices may be placed for reprocessing. One skilled the art will recognize that the reprocessing chamber 120 may be of any size or configuration necessary to hold and/or to position within the one or more medical devices. For example, in an automated endoscopic reprocessing system, the basin may be configured to hold an endoscope to be reprocessed.

In one or more embodiments, the reprocessing chamber 120 may be used to clean, disinfect and/or rinse the one or more medical devices positioned therein. For example, such medical devices (e.g., an endoscope, endoscope caps, and similar tubing for the endoscopes; polyp traps, etc.) may be reprocessed in one or more different manners. For example, an endoscope may include a fitting configured to be connected to a reprocessor fitting of the reprocessing apparatus. In such a manner, the reprocessing apparatus 100 may be fluidly connected to the medical device to provide a flushing fluid therethrough. In at least one embodiment, the reprocessing apparatus 100 includes a reprocessor fitting 122 through which at least one fluid may be provided under control of the reprocessing apparatus 100 (e.g., the reprocessing system 100 may include a control system 123 including one or more processors suitable for controlling the supply of cleaning solution, disinfecting solution, rinsing solution, etc.). The reprocessor fitting 122 is connectable to the cleaning system 10 for use in cleaning one or more components used in a medical process as further described herein.

Further, in one or more embodiments, the reprocessing apparatus 100 may also be used to clean one or more medical devices in an alternate manner to flushing fluid therethrough. For example, the one or more medical devices may be positioned within the reprocessing chamber 120 (e.g., a basin) and fluids may be provided within the reprocessing chamber 120 to clean, disinfect, and/or rinse the medical devices (e.g., an endoscope) positioned therein. For example, one or more fluids may be provided within the reprocessing chamber 120 by spraying fluids into the reprocessing chamber 120 (not shown), by filling the reprocessing chamber 120 from one or more ports located within the reprocessing chamber 120 (not shown), or by any other technique known to be used in automated reprocessing systems, such as, for example, in an automated endoscopic reprocessor. As such, automated reprocessing systems, such as generally described with reference to reprocessing apparatus 100, may include a reprocessor fitting through which a cleaning solution, a disinfecting solution, and/or a rinsing solution may be provided for flushing one or more portions of a medical device being reprocessed by the reprocessing apparatus 100 (e.g., to clean the inside of an endoscope), and may also include suitable apparatus to provide a cleaning solution, a disinfecting solution, and/or a rinsing solution to within the reprocessing chamber 120, for example, to clean, disinfect, and/or rinse one or more medical devices positioned within the reprocessing chamber 120 (e.g., to clean the outside of the endoscope).

As shown in FIG. 1, the reprocessor fitting 122 terminates a fluid connection line 124. The fluid connection line 124 is fluidly connected to one or more fluid sources 126 (e.g., sources that may be part of or separate from the reprocessing apparatus 100). The provision of fluid from the one or more fluid sources 126, such as, for example, a cleaning solution source, a disinfectant source, and a rinse source, is controlled by a control apparatus 123 (e.g., one or more processing apparatus commonly used in reprocessors such as those indicated as being known and/or available herein).

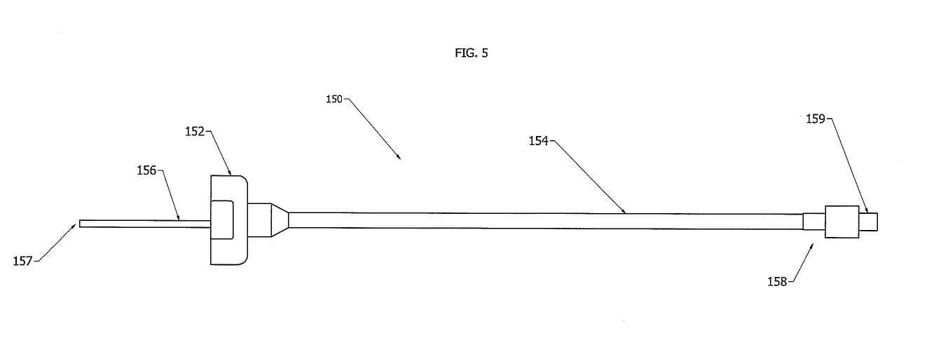

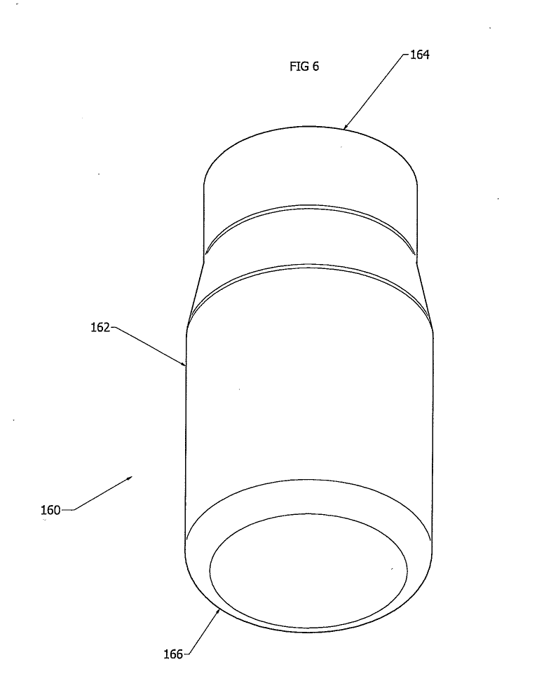

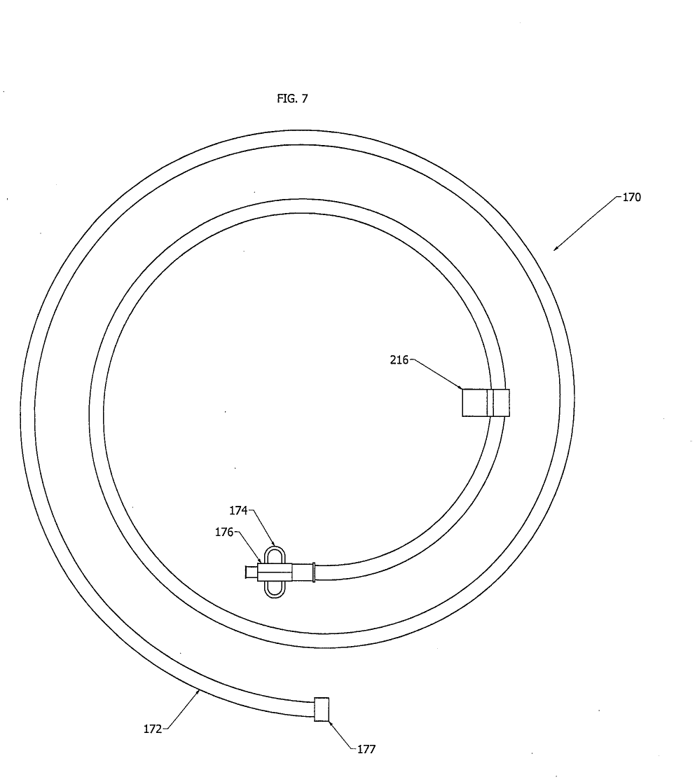

The cleaning system 10 shown generally in FIG. 1 is for use with the reprocessing apparatus 100. The cleaning system 10, at least in one embodiment, provides the ability to clean one or more components used in a medical procedure. These various components may include closable components 130 (e.g., those having a closed defined volume such as a water bottle) and flow-through components 132 (e.g., those in which a flow of fluid may be provided through a portion thereof, such as irrigation tubes and/or water bottle caps having tubes provided therethrough) as shown generally in FIG. 1. For example, as described herein, in an endoscopy procedure, various components may be used including, for example, a water bottle, a water bottle cap, an irrigation tube, a bottle base, an O-ring, etc. In one or more embodiments as described herein, the cleaning system 10 may be used with an automated endoscopic reprocessor to clean one or more components used in an endoscopic procedure including but not limited to one or more water bottles, one or more water bottle caps, and one more irrigation tubes. As used herein, the term cleaning when used in conjunction with the cleaning systems described herein refers to any type of cleaning processes, including, but not limited to, disinfecting, rinsing, alcohol processing (e.g., such as alcohol drying), sterilizing, and/or any other like processes. Further, cleaning may also include reprocessing, and in many cases the terms cleaning and reprocessing are used interchangeably herein. One or more of such components of a water bottle set are shown generally in FIGS. 5-7; FIG. 5 showing one embodiment of a water bottle cap 150, FIG. 6 showing one embodiment of a water bottle 160, and FIG. 7 showing one embodiment of an irrigation tube 170. Such water bottle and tube sets are available from, for example, Olympus, Inc., Pentax Inc., and Fujinon Inc. For example, such components are available from Olympus under the trade designation of MAJ 901 and MAJ 855.

The exemplary water bottle 160 shown in FIG. 6 includes a body of material 162 extending from an open end 164 to a closed end 166. The water bottle 160 is merely one exemplary embodiment of a water bottle usable, for example, in an endoscopic procedure. As shown in FIG. 5, the water bottle cap 150 may include a closure portion 152 for closing water bottle 160 shown in FIG. 6. The water bottle cap 150 may further include an outer tube portion 154 and an inner tube portion 156 positioned within a channel of the outer tube portion 154. The tube portions 154 and 156 are terminated at a distal end 158 by a tube fitting 159 connectable to an endoscopic system used in an endoscopic procedure (e.g., compatible with a mating connector fitting thereof). The tube portion 156 at proximal end 157 is a portion which is inserted within water bottle 160 when the water bottle cap closure portion 152 is used to close the water bottle 160. The water bottle cap 150 as shown in FIG. 5 is merely one exemplary embodiment of a cap usable to close a water bottle used in an endoscopic procedure. The irrigation tube 170 shown in FIG. 7, extends, for example, from a first end 172 terminating at a tube fitting 177 connectable to an endoscopic system used in an endoscopic procedure (e.g., compatible with a mating connector fitting thereof) to a second end 174 terminating at a tube fitting 176 connectable to a pump end of a water supply (e.g., a water bottle, etc.). One skilled in the art will recognize that various configurations of such components (e.g., water bottle sets or individual components thereof) usable in the endoscopic procedure may be cleaned using the cleaning system 10 as described herein. In no manner is the present disclosure limited to use for cleaning only those components described herein. Rather, the present disclosure contemplates use of the cleaning system 10 with any component used in a medical procedure where reprocessing of such a component is beneficial.

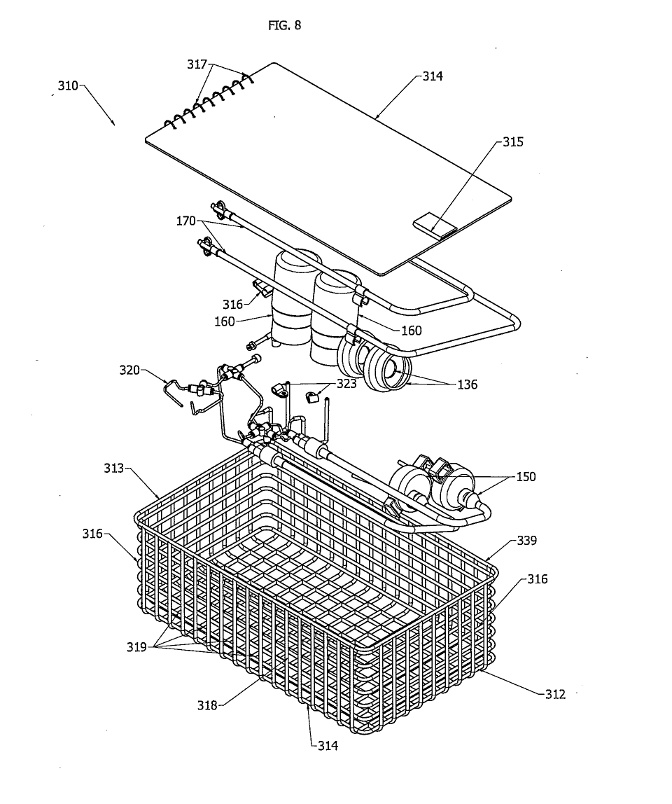

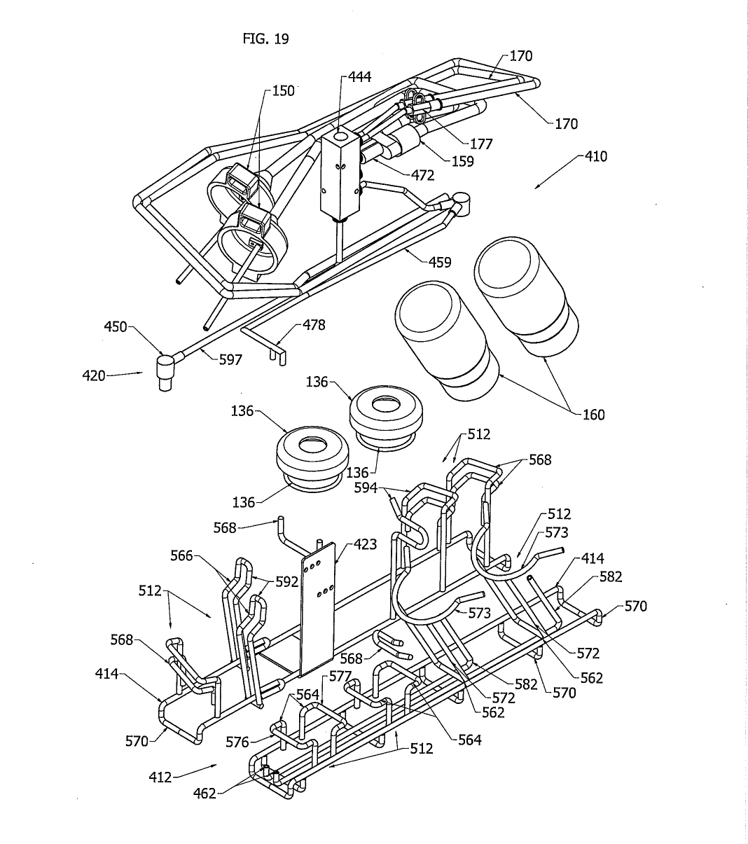

The cleaning system 10 generally shown in FIG. 1 includes a container 12 (i.e., a holding apparatus, such as a walled container as described herein with reference to FIGS. 8-18 or a non-walled container as described herein with reference to FIGS. 19-27) to receive (e.g., inside or outside a walled container by positioning or attaching, in a holding structure of a non-walled container by retaining elements, etc.) the one or more components to be cleaned (e.g., reprocessed). For example, a walled container as described herein refers to a container or holding apparatus that includes one or more walls (e.g., structure extending about or from a bottom defining a boundary) to enclose or partially enclose a volume into which one or more components may be placed (e.g., the walls may extend at least partially or entirely about one or more components). Unlike a walled container, a non-walled container refers to a container or holding apparatus that lacks such boundary defining walls that enclose or partially enclose a volume but which includes one or more holding structures (e.g., various holding structures formed as at least one rack apparatus to receive and hold one or more components, wherein such components are held in position without the use of walls of material). For example, the non-walled container may be provided as a framework of elements for holding the one or more components at particular locations (e.g., the non-walled container may be configured as at least one rack apparatus including a bottom structure relative to which one or more of the plurality of holding structures are arranged to receive the one or more components at particular locations of the at least one rack apparatus).

In one of more embodiments, the container 12 may be a walled container (i.e., a holding apparatus) that includes at least a bottom and one or more sidewalls. At least portions of the bottom and the one or more sidewalls may include a network of material providing a plurality of openings to allow fluid to pass therethrough to contact the one or more components. For example, when the container is positioned within the reprocessing chamber 120, fluid is allowed to enter the walled container 12 through the plurality of openings to contact the one or more components positioned within the walled container 12. At least in one embodiment, for example, when the reprocessing chamber 120 is filled with fluid under control of the reprocessing apparatus 100 such fluid is allowed to enter the walled container 12 and contact the one or more components for cleaning, disinfecting, and/or rinsing.

The network of material providing the plurality of openings may include and/or be formed in any suitable manner. In one or more embodiments, the network of material providing such openings may include a wire grid, a wire mesh, a material defining a plurality of openings, metal structure defining a plurality of openings, plastic or molded plastic defining a plurality of openings, etc. Any suitable material may be used to provide the network of material, such as, for example, stainless steel, plastic, molded plastic, solid material defining one or more openings, etc. In one or more embodiments, the network of material may be integral as compared to formed of various portions or sections coupled together.

In one of more embodiments, the container 12 may be a non-walled container (i.e., a holding apparatus) to receive the one or more components. For example, the non-walled container may include a plurality of holding structures, wherein each of the plurality of holding structures may receive at least one component of the one or more components and maintain the at least one component in a position when fluid is allowed to contact the one or more components. For example, the plurality of holding structures may form a rack structure (e.g., an integrated rack or various rack portions) for receiving one or more components and maintain such components in a position thereon when fluid is allowed to contact the one or more components.

For example, the plurality of holding structures may be provided in any variety of configurations to retain the components during cleaning. For example, the plurality of holding structures may include one or more retaining elements that are insertable into at least a portion of at least one component of the one or more components (e.g., a curved element inserted into an opening defined by a portion of the component, such as an opening in a bottle cap) to maintain the at least one component in a position when fluid is allowed to contact the at least one component. Further, for example, the plurality of holding structures may include one or more retaining elements that engage at least a surface portion of at least one component of the one or more components (e.g., a clamping element that may be flexed from a normal state, to engage an outer surface of a component, such as a water bottle) to maintain the at least one component in a position when fluid is allowed to contact the at least one component. Further, for example, the plurality of holding structures may include one or more retaining elements into which at least a portion of at least one component of the one or more components is insertable (e.g., a component inserted into a retaining structure defining a space with the retaining structure holding the component in place) to maintain the at least one component in a position when fluid is allowed to contact the at least one component. Any number of different retaining structures may be used to provide the holding function and the structures provided herein are not to be taken as limiting on the holding structures suitable to provide such functionality.

In one or more embodiments, the container 12 is a non-collapsible container sized to be received within a reprocessing chamber 120. As used herein, the term non-collapsible in combination with the term container refers to a container being self-supporting such that the container is suitable to provide a draining function (e.g., gravity drain). For example, the bottom and one or more sidewalls of a walled container may be formed as wire grids coupled together to form a self-standing container defining a volume therein. In a non-walled container, for example, the holding structures (e.g., formed of one or more tubes or wires; rigid and/or flexible) may be coupled together to form a self-standing container (e.g., a rack structure).

Further, in one or more embodiments, the bottom of the container 12 may be malleable or lack a rigid planar bottom structure (e.g., stand-off portions may be used) such that the bottom forms or is otherwise adapted to be effectively received by a structure in the reprocessing chamber 120 of the reprocessing apparatus 100 on which it is to be received. For example, a basin type reprocessing chamber of an automated endoscopic reprocessing apparatus may include a non-planar bottom. The malleability of the bottom of the container may allow the container 12 to form to such a non-planar bottom. Further, one or more stand-off structures may be used to separate the components from being directly in contact with the non-planar bottom of the reprocessing chamber. As not all automated endoscopic reprocessing apparatus include the same type of structure upon which the container 12 is to be positioned, such configurations provide an effective structure such that the container 12 may be used with any number of automated endoscopic reprocessing apparatus which may not have similar structures upon which the container would be positioned.

Still further, in one or more embodiments, the container 12 may further include a cover. For example, the cover may be formed of a similar material to the remainder of the container 12 or may be formed of a material completely different from the network of material. In one or more embodiments, the cover may be a solid cover, the cover may be formed of a network of material defining openings, or the cover may be provided by any other structure suitable to retain the one or more components positioned within the container 12 during reprocessing. For example, during reprocessing of the one or more components positioned within the container 12 by the reprocessing apparatus 100, fluid may be provided at such pressures causing one or more components (e.g., water bottles, washers, etc.) to move within the container 12 (e.g., possibly being ejected from the container 12 if a cover is not present).

The cleaning system 10 further includes a manifold apparatus 14 configured to be coupled to the container 12 (e.g., attached at one or more portions of the container, positioned at least partly within the container 12 unattached from the container but maintained in a particular location in or relative to the container, received within the container 12 at a location, etc.). The manifold apparatus 14 may be coupled to the container 12 using any suitable coupling apparatus such as, for example, clips, fasteners, clamps, soldered connections (e.g., by welding), adhesive connections (e.g., using glues or adhesives), structure of the manifold apparatus 14 and/or the container 12 that mates or otherwise maintains the manifold apparatus 14 (or portions thereof) in position within the container 12 (e.g., one or more portions of the wire grid mating with a tube portion of the manifold apparatus, one or more portions of the manifold apparatus fitting (e.g., press fit) within structure of the container 12, etc.), material of the container simply maintaining the manifold apparatus 14 or portions thereof in a location in the container 12 (e.g., friction coupling between the manifold apparatus 14 and container 12), etc.

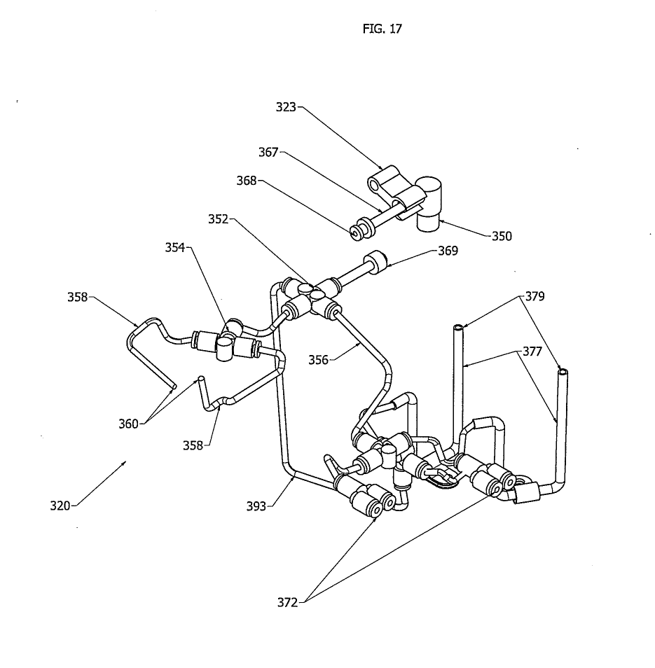

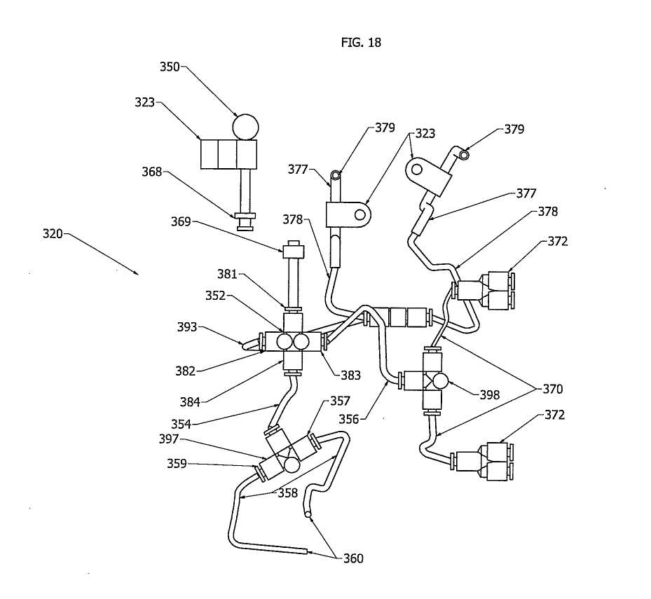

The manifold apparatus 14 may include an inlet connection 16 connectable to the reprocessor fitting 122 of the reprocessing apparatus 100 to receive at least one fluid from the reprocessing apparatus 100 (e.g., a cleaning solution, a disinfecting solution, and a rinsing solution, etc.). The inlet connection 16, for example, may include a connection compatible with (e.g., adapted to mate with) the reprocessor fitting 122. For example, a particular reprocessing apparatus 100 may include a defined reprocessor fitting 122, and as such, in one or more embodiments, the inlet connection 16 may be compatible to provide a fluid tight connection between the inlet connection 16 and the reprocessor fitting 122 (e.g., the inlet connection may include a part available from the reprocessor manufacturer which mates with the reprocessor fitting 122). For example, in one embodiment, the reprocessor fitting 122 may include one or more of the following types of fittings: luer lock fittings, threaded fittings, soldered fittings, welded permanent components that connect to inlet connection 16 (e.g., are easily attached and detached), etc.; and the inlet connection 16 would provide a connection compatible therewith. For example, the reprocessor fitting 122 and the inlet connection 16 may be mating components of a luer lock device, a threaded device, a pressure fitting, or any other suitable fitting such as those that provide a fluid tight connection. Further, for example, the inlet connection may include a fitting compatible with the reprocessor fitting 122, a male/female luer lock fitting configured to mate with another male/female luer lock fitting of the manifold apparatus, and a portion of tubing therebetween (e.g., such that it may be easily connected to the reprocessor fitting 122; see FIGS. 17-18).

Further, the manifold apparatus 14 may include one or more outlets 18 configured to provide at least one fluid into or through one or more components 130 and 132 as shown generally in FIG. 1. In one of more embodiments, the one or more outlets 18 may include at least one outlet connection connectable to (e.g., compatible with to provide a fluid tight connection) a component fitting of at least one of the one or more components. For example, as shown in FIG. 1, the one or more components may include a closed component 130 (e.g., a water bottle 160) and the manifold apparatus 14 may include one or more manifold tube portions 19 fluidly connected in the path from the inlet connection 16 to the one or more outlets 18. For example, at least one of the one or more manifold tube portions 23 may include an outlet insertable into an interior volume of the at least one closed component 130. For example, the at least one manifold tube portion may include an outlet terminating a tube portion extending and pointing away from the bottom of the container 12 when the manifold apparatus 14 is coupled to the container 12 such that an open end of the closed component (e.g., water bottle) is positionable onto the tube and/or the open end is insertable into the closed component.

Further, for example, the one or more components may include a flow-through component 132 as shown in FIG. 1 that includes a defined tube fitting 25. In one or more embodiments, the manifold apparatus 14 may include an outlet connection 18 connectable to the defined tube fitting 25 of the component 132 (e.g., compatible therewith to provide a fluid tight connection). For example, in one or more embodiments, the one or more components may include at least one water bottle cap used in an endoscopic procedure wherein the water bottle cap includes at least one tube therethrough terminating at a tube fitting connectable to an endoscopic system used in an endoscopic procedure. In such a case, the at least one outlet connection 18 of the one or more outlets 19 of the manifold apparatus 14 may be connectable to a tube fitting terminating the at least one tube extending through the water bottle (e.g., compatible with the tube fitting to provide a fluid tight connection). Further, for example, the one or more components may include at least one irrigation tube used in an endoscopic procedure, wherein the irrigation tube extends between a first end and a second end with at least one of the ends terminating at a tube fitting connectable to an endoscopic system used in an endoscopic procedure. In such case, the at least one outlet connection 18 of the one or more outlets 19 of the manifold apparatus 14 may be connectable to the tube fitting of the at least one irrigation tube (e.g., compatible with the tube fitting to provide a fluid tight connection so fluid can be flushed therethrough).

In other words, as shown in FIG. 1, the one or more outlets 18 may be any suitable configuration to provide at least one fluid into and/or through one or more components positioned within the container 12. In one or more embodiments, the outlets 18 may be configured as outlet connections compatible with the fittings of the one or more components received by the container 12 to provide a flushing fluid through at least a portion of the one or more components or may be configured without a connection so as to simply provide, for example, a spray of at least one fluid into a closed container. Still further, the manifold apparatus 14 may include any number components (e.g., rigid or flexible) such as tube portions, input/output connections (e.g., tee connections), or any other structure, between the inlet connection 16 and the outlets 18 suitable to provide fluid (e.g., liquid or gas) from the inlet connection 16 to the one or more outlets 18.

Further, an exemplary method of cleaning one or more components used in a medical procedure may also be described with reference to FIG. 1. For example, such a cleaning method may include providing a reprocessing apparatus 100 including a reprocessing chamber 120 and a reprocessor fitting 122 through which at least one fluid is provided (e.g., a cleaning solution, a disinfecting solution, a rinsing solution, etc.). Further, a cleaning system 10 such as described herein may also be provided, including, for example, a container 12 (e.g., a walled or non-walled container) and a manifold apparatus 14. The method may further include positioning at least one of the one or more components 130, 132 relative to the container 12 (e.g., into the walled container 12, relative to one or more holding structures of a non-walled container, wrapped about a walled container, snapped into a retaining structure, inserted into a retaining structure, clamped into a retaining structure, positioning on a retaining structure, connecting at least one outlet connection 18 to a component fitting 25 of at least one of the one or more components 132, etc.) and positioning the container 12 (e.g., along with at least portions of the manifold) into the reprocessing chamber 120 (e.g., which may include connecting the inlet connection 16 to the reprocessor fitting 122 of the reprocessing apparatus 100). In one or more embodiments, one or more of the components may also be positioned outside of a volume defined by the interior of a walled container (e.g., such as an irrigation tube attached and/or wrapped about the walled container using attachment apparatus, such as clips, hooks, etc.). At least one fluid may then be provided into or through at least one of the one or more components 130, 132 held by the container 12 and positioned into the reprocessing chamber 120.

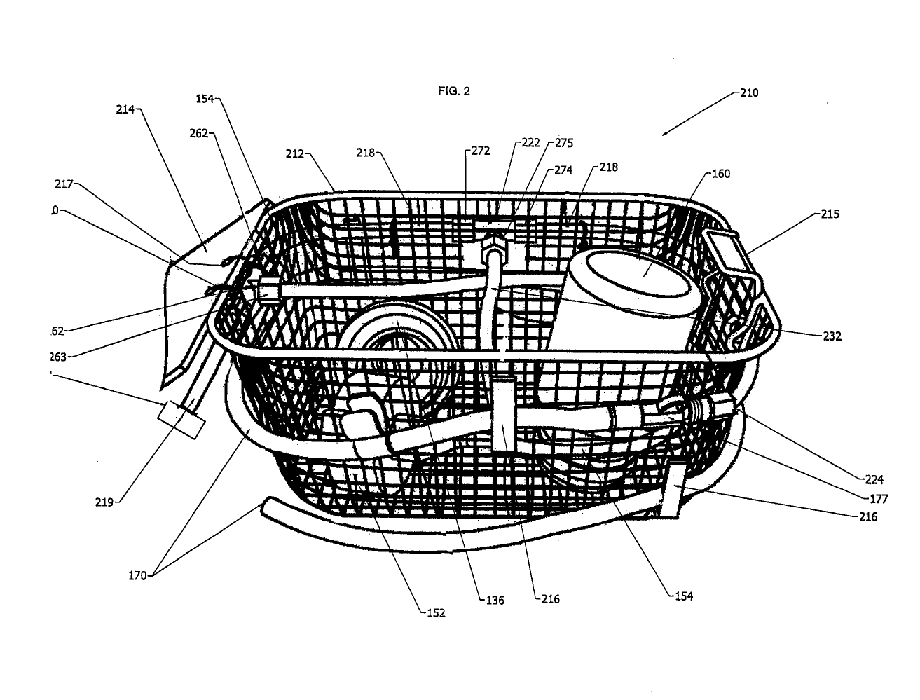

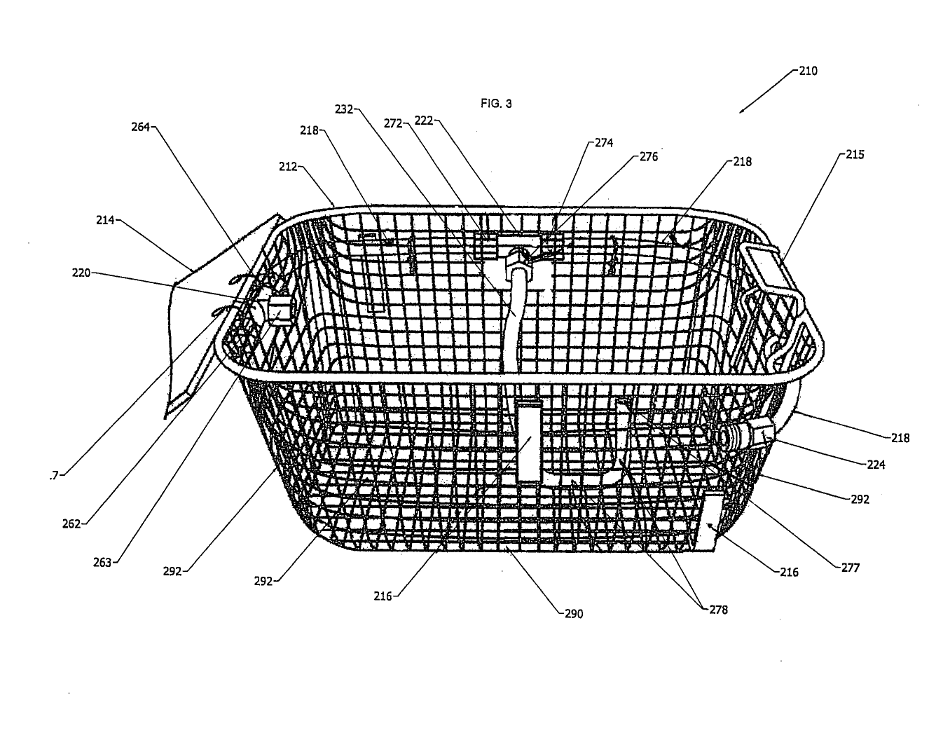

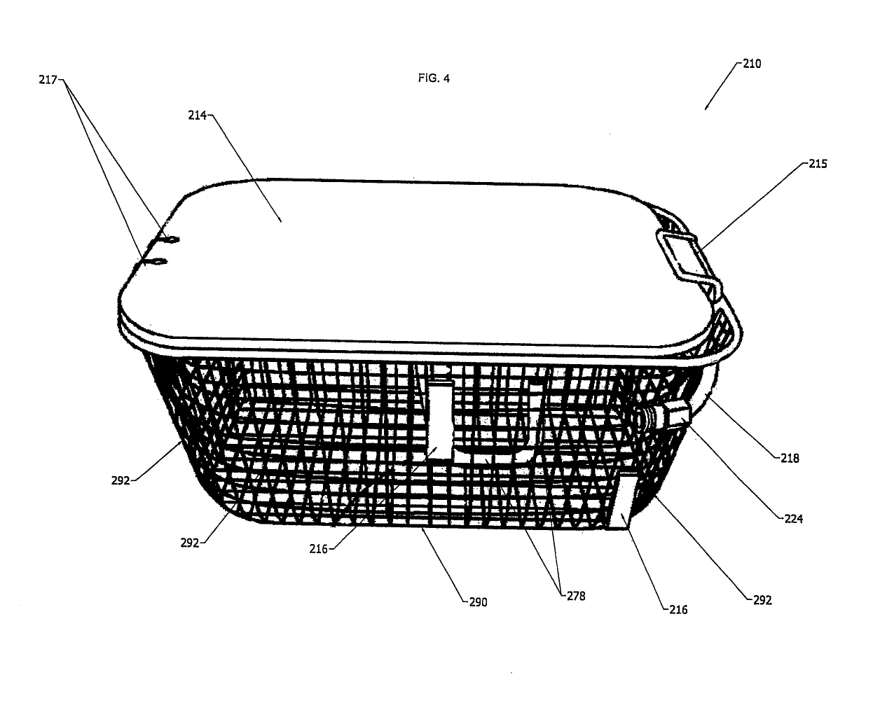

One exemplary embodiment of a cleaning system 210 (e.g., a gastrointestinal (GI) endoscopic irrigation cleaning system), such as shown more generally by the cleaning system 10 in FIG. 1, shall be described with reference to FIGS. 2-4. FIG. 2 shows a perspective view of the cleaning system 210 with endoscopic components to be cleaned, FIG. 3 shows a perspective view of the cleaning system 210 in an empty state, and FIG. 4 shows a perspective view of the cleaning system 210 with a lid 214 thereof closed. The exemplary cleaning system 210 is, for example, provided for cleaning one or more components such as, for example, those shown in FIGS. 5-7 (e.g., a water bottle set including at least a water bottle 160, a water bottle cap 150, and an irrigation tube 170). Also shown in FIG. 2 are an O-ring and base holder 136 which may also be a part of, or components of, the water bottle set to be cleaned.

Current GI endoscopic irrigation cleaning processes are typically limited to the manufacturer's guidelines which may be difficult to standardize and/or the performance of which may be difficult to repeat performance across different technicians. Another method does away with cleaning and allows the water bottle, cap, and tubing to be disposable. This method is, however, not environmentally friendly and results in additional costs. The present disclosure provides a cleaning system that allows for high level disinfection to be standardized and repeated correctly every time. The present disclosure may save the medical community revenue and may keep the trend away from filling landfills with disposable products.

Referring to FIGS. 2-7, the cleaning system 210 may include a container (e.g., a walled container) such as cleaning basket 212. The cleaning basket 212 may be made of, for example, stainless steel, plastic, or the like. The basket 212 may or may not include the cover or lid 214. An inlet tubing 219 (e.g., part of a manifold apparatus 218 that provides fluid to the one or more components positioned in or about the basket or container and that terminates at an inlet connection or fitting 237 compatible with a reprocessor fitting of an AER) may provide disinfectant, rinse water, air, or the like (e.g., a fluid) to the cleaning system 210. For example, the inlet tubing 219 may be connected to an automated endoscopic reprocessor (AER) and the basket 212 may be positioned in the AER (e.g., in a reprocessing chamber thereof) during the cleaning process. Any suitable connection configuration of the manifold apparatus 218 to the AER may be used which, for example, provides an inlet connection compatible with a predefined reprocessor fitting of the AER. For example, a reprocessor fitting of a reprocessing apparatus is available for providing a fluid tight connection to an inlet connection of manifold apparatus 218 (e.g., an inlet connection compatible with the reprocessor fitting of the AER; which may be a part manufactured by the AER manufacturer for use with an endoscope cleaning process but used on the manifold apparatus 218 as part of the inlet connection thereof). The basket 212 may include a bottom 290 and one or more side walls 292 which form and define an interior volume into which one or more components may be positioned provide structure onto which one or more components may be attached or wrapped about, etc.

A first tee 220 (e.g., of the manifold apparatus 218) may include an inlet port 262 connected to the inlet tubing 219 and may provide a connection 263 to water bottle tubing 154 (e.g., provide an outlet connection compatible with and connectable to a tube fitting 159 terminating the one or more tubes 154, 156 extending through the water bottle cap 150). In some embodiments, the water bottle cap 150 may be placed in the basket 212, with its ending tube 157 sticking out of the bottom of the basket 212. Fluid, such as disinfectant, may flow through the inlet fitting 237 (e.g., which is compatible with an AER reprocessor fitting) and inlet tubing 219, through the first tee 220, and through the water bottle cap tubing 154, 156. Because of the position of the cap 150, when a cleaning cycle is complete, gravity may drain residual water from the tubing 154, 156.

From another outlet port 264 of the first tee 220, a tube connector manifold portion of manifold apparatus 218 may wrap around one side of the basket 212 to a second tee 222. The second tee 222 includes an inlet port 272 fluidly connected by a tube to the outlet port 264, and includes first and second outlet ports 274, 276. This second tee 222 may provide a fluid feed through a water bottle cleaning tube 232 for providing fluid to within a water bottle in which the distal end of the tube 232 is positioned (e.g., the tube 232 is connected to the outlet port 276 of tee 222 by way of a fitting 275 and terminates at outlet 277 at the distal end of tube 232). This tube 232 may run into the basket 212, to the base or bottom 290 of the basket 212, and may point upward from the base or bottom 290 of the basket 212. A water bottle 160 may be placed upside down over the outlet 277 at the distal end of the tube 232 for cleaning. A sprayer (not shown) may optionally be disposed on the distal end of the tube 232 to provide a spray to the inside of the bottle 160.

From the other outlet 274 of the second tee 222, a tube connector manifold portion of manifold apparatus 218 may continue and wrap around the basket to terminate at a fitting 224. This fitting 224 may be adapted to attach to (e.g., be compatible with) the tube fitting 177 at the first end 172 of the irrigation tube 170 shown in FIG. 7. The irrigation tube 170 may wrap around the outside of the basket 212, being secured to the basket 212 with a plurality of hooks 216. For example, a first one of the hooks 216 may position the irrigation tube 170 slightly below the fitting 224. Each subsequent hook 216 may be slightly lower (e.g., closer to the bottom of the basket 212). This configuration may permit the irrigation tube 170 to drain due to gravity (i.e., gravity drain) after the cleaning process.

The following describes exemplary process steps for cleaning various GI endoscopy components, such as the water bottle 160, water bottle cap 150, irrigation tube 170, base holder or bottle base and O-ring 136, etc. For example, the process may include connection of an inlet connection of the manifold apparatus 218 to a reprocessor fitting of an AER. For example, this may include taking tube inlet 219 (e.g., including an inlet connection fitting 237) and hooking this up to connector tubing from the AER (e.g., the tube inlet 219 including a fitting that is compatible with or mates with a reprocessor fitting of the AER). The basket 212 (e.g., a stainless steel basket) may be properly positioned in the AER. The water bottle cap 150 is positioned with the closure portion 152 in the downward position with ending tube 157 sticking out of the bottom of the basket 212 (e.g., the stainless steel basket). The main tubing 154, 156 is run up to the connection point 263 at the first tee 220. This should allow the water bottle cap tubing 154, 156 to be positioned such that gravity will allow drainage to complete the process. The water bottle 160 is positioned onto the tube outlet 277 of the tube portion 232 (e.g., a stainless steel stem portion 278) in the basket 212. The water bottle 160 is positioned upside down onto the tube outlet 277 of the tube stem portion 278.

Further, the tube fitting 177 of the irrigation tube 170 is connected to fitting 224. The length of the irrigation tube 170 is wrapped around the outside of the basket 212 setting the tube 170 in the side hooks 216 on one or more sides (e.g., all sides) of the basket 212. The irrigation tube 170 may flow in gravitational direction as it is hooked to the side of the basket 212. Any other components, such as any O-rings and base holders 136, and the like are put at the bottom of basket 212. Such items may sit in any open area because when the lid or cover 214 (e.g., a stainless steel lid) is closed and latched shut they will be retained in the basket 212 (e.g., lid 214 may be hinged 217 at one side of the basket 212 and a latch 215 may be provided on the opposite side to latch or lock the lid 214 in place).

In one or more embodiments, with at least the water bottle, water bottle cap, and irrigation tube in the basket 212, and the basket 212 in the reprocessing chamber, the AER may be operated as desired for cleaning (e.g., flushing, disinfecting, submersing, rinsing, etc.) the components in the basket 212. For example, a user may simply follow the manufacturer's guidelines for disinfecting rinses or reprocessing. For example, air may be run thru the lines to get out as much or all of the fluids therein. All of the irrigation original equipment manufacturer (OEM) products (e.g., the water bottle, the water bottle cap, the irrigation tube, bottle based or base holders, washers, O-rings, cap assemblies, jars, etc.) may continue to air dry as needed. One skilled it the art will recognize that process steps of this exemplary method, as well as other methods described herein, are not limited by time sequence in which they are described. For example, the various process steps may be carried out in any order possible to provide the functionality described.

The single basket cleaning system configuration 210 described herein can be doubled, tripled, or the like, as space may permit (e.g., two or more sets of water bottles, caps and irrigation tube may be cleaned (e.g., reprocessed) at a time, for example, during a single reprocessing operation of an AER. This allows the facility to choose which size configuration they would like to use. In one or more embodiments, either multiple containers (e.g., walled containers such as baskets, non-walled containers, etc.) may be used or alternatively the size of the container may be configured to receive more than a single water bottle set (e.g., a single container may be configured to receive two or more water bottles, two or more caps, and two or more irrigation tubes). In one or more embodiments, the size may be limited by the configuration of the reprocessing chamber into which the container is placed.

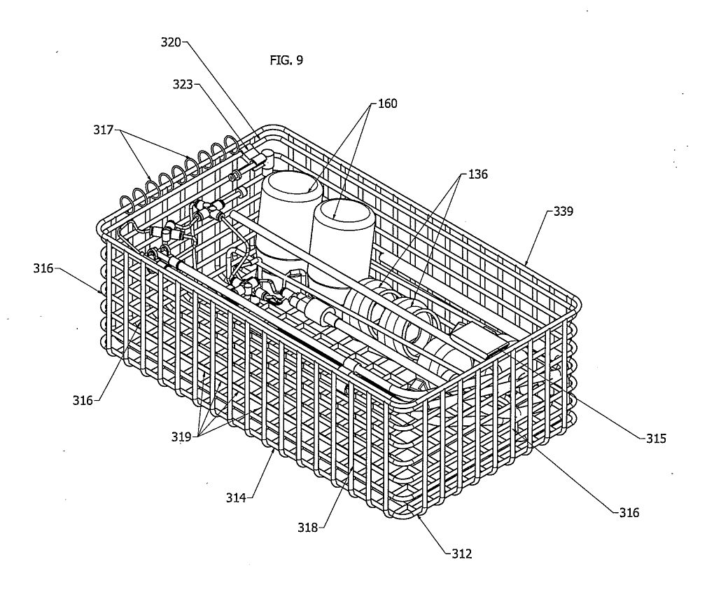

Another exemplary embodiment of a cleaning system 310 (e.g., a gastrointestinal (GI) endoscopic irrigation cleaning system), such as shown more generally in FIG. 1, shall be described with reference to FIGS. 8-18. FIG. 8 shows an exploded perspective view of the cleaning system 310 including the endoscopic components to be cleaned. FIG. 9 shows a perspective view of the cleaning system 310 with a lid 314 removed and with endoscopic components (e.g., water bottles 160, water bottle caps 150, and irrigation tubes 170, as well as bottle bases and O-rings 136) positioned in a container 312 (e.g., a walled container such as a wash basket formed at least in part of open matrix material) to be cleaned. Unlike the embodiment described with reference to FIGS. 2-4, the cleaning system 310 is configured to clean multiple water bottle sets.

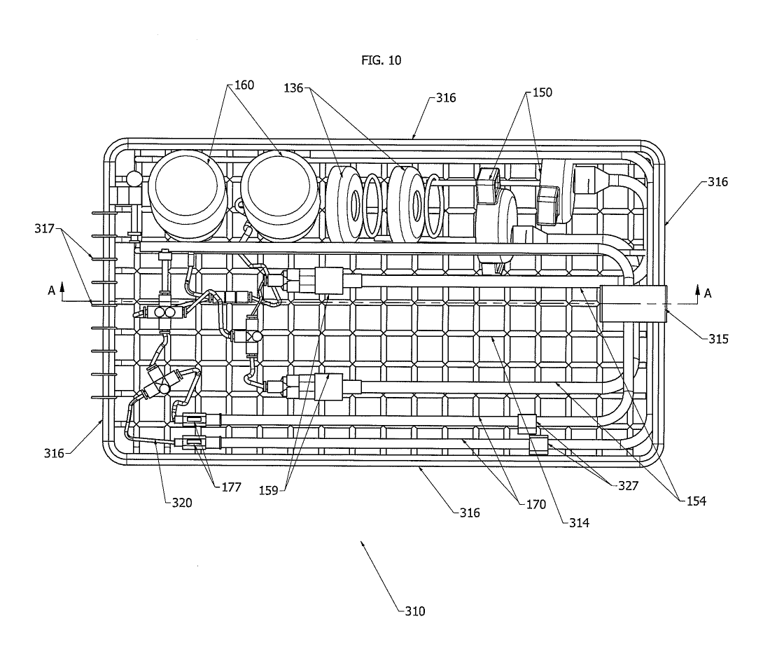

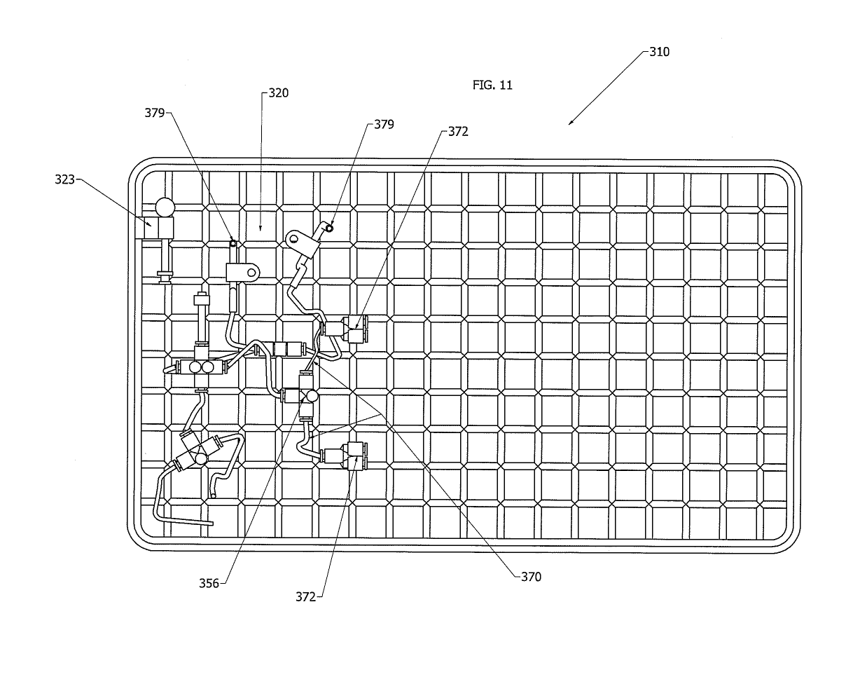

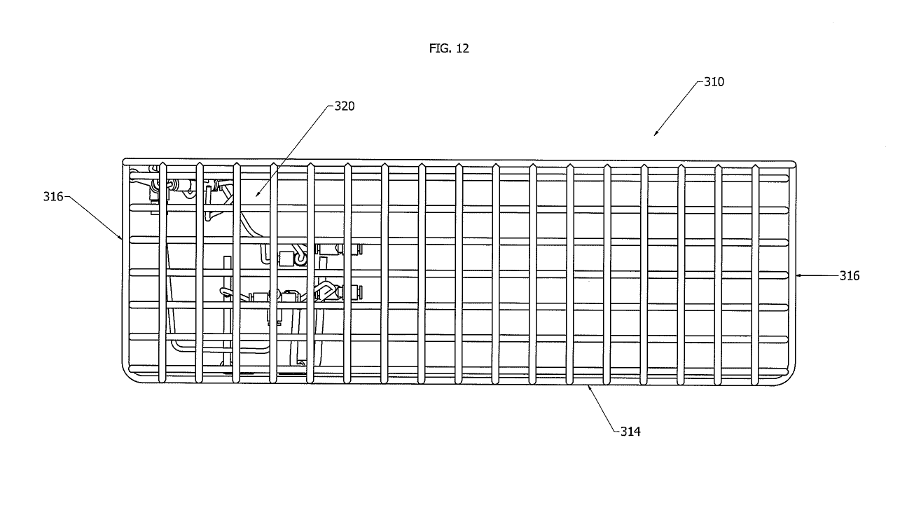

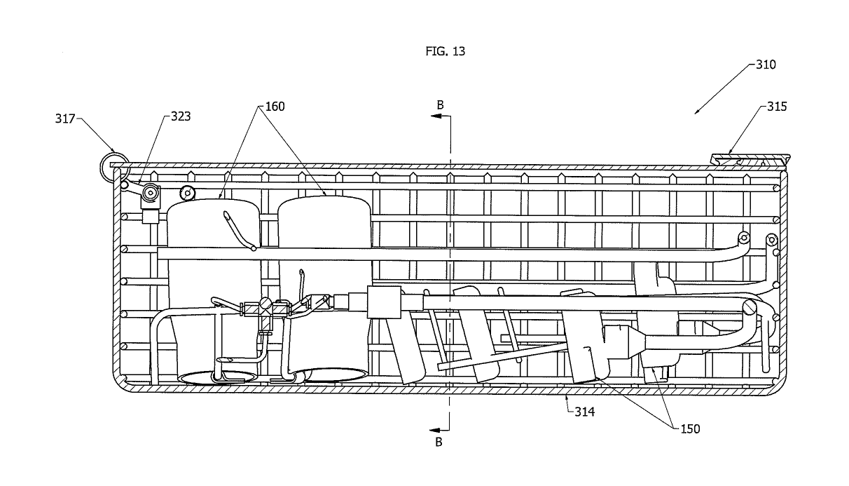

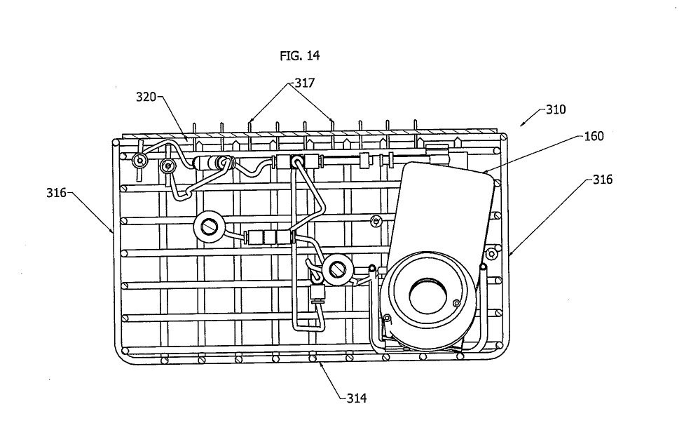

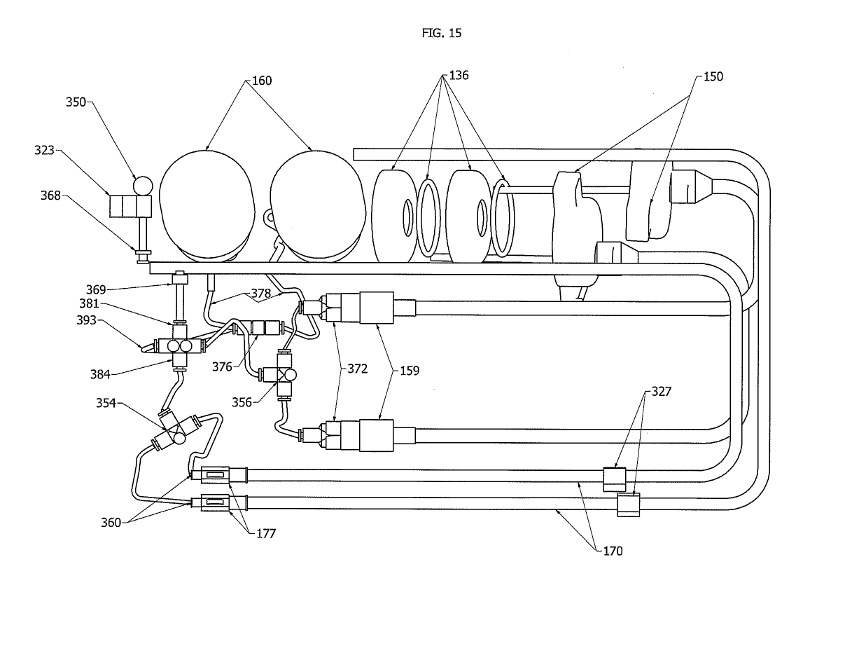

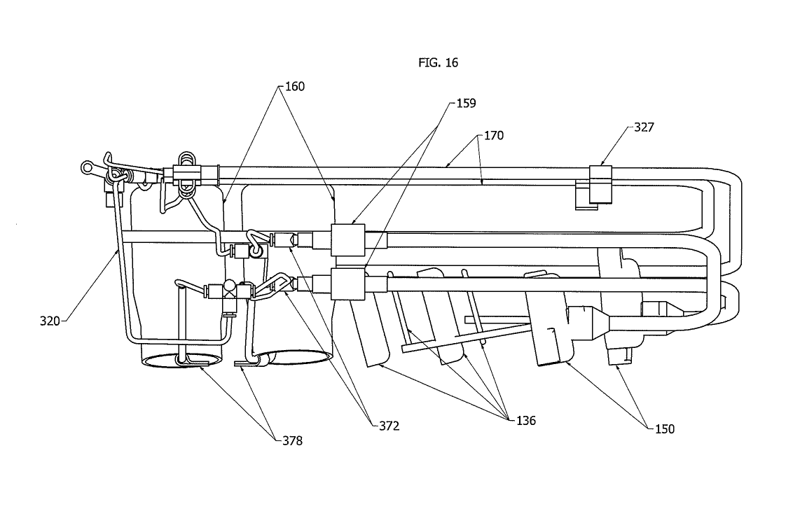

FIG. 10 shows a top view of the cleaning system 310 including the endoscopic components to be cleaned, while FIGS. 11 and 12 show a top view and a side view, respectively, of the cleaning system 310 with endoscopic components therein removed. FIG. 13 shows a cross-section view of the cleaning system 310 taken along line A-A as shown in FIG. 10 and FIG. 14 shows a cross-section view of the cleaning system 310 taken along line B-B as shown in FIG. 13. Still further, FIGS. 15 and 16 show a top view and side view, respectively, of an exemplary manifold apparatus 320 (e.g., a hose assembly including various tubes and connectors) that may be used in the cleaning system 310 with endoscopic components connected or positioned for cleaning, while FIGS. 17 and 18 show a side perspective view and a top view, respectively, of the exemplary manifold apparatus 320 used in the cleaning system 310 with the endoscopic components removed.

The walled container 312 of the cleaning system 310 as shown in FIGS. 8-18 includes a bottom 314 and one or more sidewalls 316. In particular as shown in such figures, the bottom 314 is of a generally rectangular configuration with four sidewalls 316 extending upward therefrom. At least the one or more sidewalls 316 have structure providing a rigidity to make the container 312 non-collapsible (e.g., self-supporting or free-standing). The container 312, at least in one embodiment, includes portions formed of wire grid material 318. For example, both the bottom 314 and the sidewalls 316 are formed of a network of material defining a plurality of openings 319 so as to allow fluids to enter the interior of the container 312 as well as drain by gravity therefrom. In one or more embodiments, the entire container 312 is formed of a wire grid structure and includes a rim 313 defining an opening to the interior thereof. As described herein, the network of material defining the plurality of openings 319 may include any suitable type of material that provides openings to the interior of the container 312 allowing fluid flow into the interior volume and drain therefrom, including, but not limited to, mesh materials, wire grid materials, grid structure formed of polymers, metals defining openings, plastic defining openings, molded plastic defining openings, etc. Further, the container 312 may be formed of any suitable materials including, for example, stainless steel, plastic, molded plastic, noncorrosive metals, etc. A cover or lid 314 may close the opening to the interior of the container 312. Any components, such as any O-rings and base holders 136, or water bottles 160, and the like in the container 312 will be retained therein when the lid or cover 314 (e.g., a stainless steel lid, plastic lid) is closed and latched shut (e.g., lid 314 may be hinged 317 at one side of the basket 312 and a latch 315 may be provided on the opposite side to latch or lock the lid 314 in place on rim 313).

The container 312 is sized, at least in one embodiment, to be positioned within a reprocessing chamber of a reprocessing apparatus (e.g., the basin of an AER apparatus). The container 312 may be of any size or shape, such as, but clearly not limited to, a rectangular shape, cylindrical shape, a square shape, or any other polygon shape.

In one or more embodiments, the bottom 314 of the container 312 may be malleable so as to allow the bottom 314 to conform to a structure upon which it is placed. For example, when positioned within the basin of an AER apparatus, the bottom of the basin may be non-planar or include components mounted thereon. By providing a malleable bottom 314, the container 312 may be positionable in any number of reprocessing chambers where such reprocessing chambers have different types of structures upon which the container 312 is to be placed. This may assist in reducing the height profile of the container 312 when positioned in the reprocessing chamber.

In one or more embodiments, the container 312 may be provided as a single structure or as multiple structures coupled or uncoupled to one another. For example, the container 312 may be provided as two separate containers each capable of holding a different water bottle set or any other components as desired. Further, for example, the container 312 may include various structural elements within the interior of the container 312 separating one or more portions thereof from other portions. For example, the container 312 may include one or more wall grid structures to separate or partition the interior of the container 312 into various sections, such as, for example, to receive certain of the one or more components. For example, sections of the interior may be compartmentalized for receiving a water bottle (e.g., holding the bottle upright), the water bottle cap and/or portions thereof, etc.

The manifold apparatus 320 of the cleaning system 310 as shown in FIGS. 8-18, at least in one embodiment, is coupled to the container 312 by one or more attachment mechanisms 323. The manifold apparatus 320 when coupled to the container 312 (e.g., a cage like container) holds the components in specific locations therein. Various portions of the manifold apparatus 320 may be attached to the container 312 (e.g., tube portions, inlet connections, etc.). For example, the one or more attachment mechanisms 323 may include any sort of fastener or process such as, but clearly not limited to, clips (e.g., P-clips), clamps, straps, locking mechanisms, welding, gluing, etc. As shown in the Figures, the attachment mechanisms 323 include clips attachable to the wire grid network of material 318 through one or more openings defined thereby at side walls 316 or the bottom 314 of the container 312. However, any suitable attachment technique may be used to effectively position the manifold apparatus 320, or any portions thereof, such that one or more components positioned within the container 312 can be connected as desired to the outlets of the manifold apparatus 320. At least in one embodiment, the manifold apparatus 320 is attached to an upper portion of one of sidewalls 316.

Perhaps as best shown in FIGS. 15-18, the exemplary manifold apparatus 320 may include any number of inlet connections, outlets, outlet connections, and/or tube portions suitable for carrying out the function of providing one or more fluids (e.g., cleaning solutions, disinfecting solutions, rinsing solutions, etc.) from the reprocessing apparatus to the one or more components being cleaned by the cleaning system 310 (e.g., a water bottle, a water bottle cap, irrigation tube, base holders, O-rings, etc.). For example, as described with reference to manifold apparatus 320 shown therein, an inlet connection 350 is provided for connection to a reprocessor fitting of a reprocessing apparatus to receive at least one fluid therefrom. The inlet connection 350 may be a connection compatible with the reprocessor fitting to provide a fluid tight connection therebetween. For example, the reprocessor fitting of a reprocessing apparatus is generally available within the reprocessing chamber for connection to, for example, an endoscope to be cleaned by the reprocessing apparatus. As such, the inlet connection 350 may be compatible with such a reprocessor fitting (e.g., the inlet connection may be a component from the reprocessor manufacturer, used to connect an endoscope, but now used as part of the manifold apparatus 320). Further, for example, the inlet connection 350 may include tubing for providing the connection to the reprocessor fitting.

In the embodiment shown in FIGS. 15-18, the inlet connection 350 (e.g., including a reprocessor manufacturer's part compatible with the reprocessor fitting of a specific AER) is connected by tubing to a luer lock female end 368. The luer lock female end 368 is configured to mate with a luer lock male end 369 connected by tubing to inlet port 381 of a distribution element 352. However, the inlet connection 350 may be connected to the distribution element 352 in any manner or configuration. This configuration shown in FIGS. 17-18 allows for easily adapting the manifold apparatus to be used with more than one reprocessor (e.g., by disconnecting the tube portion at the luer lock 368/369 and using a different tube portion having a fitting 350 compatible with a different AER). The distribution element 352 may include any number of inlet and outlet ports and be provided by any configuration of elements and tubing (e.g., a multiple port apparatus, one or more tee fittings connected together, etc.).

The distribution element 352, for example, may provide fluid from the inlet connection 350 to a first tube portion 354 via outlet port 384 and to a second tube portion 356 via outlet port 383. The first tube portion 354 may be bifurcated into two tube channels 358 each terminating at an outlet connection 360 (e.g., via a tee connector 397). Each of the outlet connections 360, for example, may be compatible for connection to the tube fitting 177 of an irrigation tube 170 for use in flushing fluid through the irrigation tube 170 during the cleaning cycle. The irrigation tubes 170 may be held at various levels of elevation by clips 327 or other attachment elements such as shown in FIG. 10 so as to position the irrigation tubes 170 for gravity draining.

The second tube portion 356, for example, may also be bifurcated into two tube channels 370 each terminating at an outlet connection 372 (e.g., via a tee connector 398). Each of the outlet connections 372, for example, may be compatible for connection to the tube fitting 159 of a water bottle cap 150 for use in flushing fluid through the tubes 154, 156 of the water bottle cap 150.

Still further, the manifold apparatus 320 may include another tube portion 393 terminating at a tee connection 376. The two outlets of the tee connection 376 provide for a bifurcation of the tube portion 393 into two tube channels 378. Each of the two tube channels 378 are shaped and/or configured to provide a structure upon which a water bottle 160 may be positioned. For example, as shown in FIG. 17-18, each of the two tube channels 378 include a distal tube portion 377 (e.g., stainless steel portions, or other structure suitable to support a water bottle in an inverted position) terminated at an open end 379. The distal tube portions 377 are provided when the manifold apparatus 320 is coupled to the container 312 such that they extend away from the bottom 314 of the container 312 (e.g., in generally a perpendicular direction to the bottom 314). One will recognize that any configuration of the tube channels 378 may be used which allow fluid to be provided into the interior of the water bottle 160 and that allow the bottles to drain by gravity.

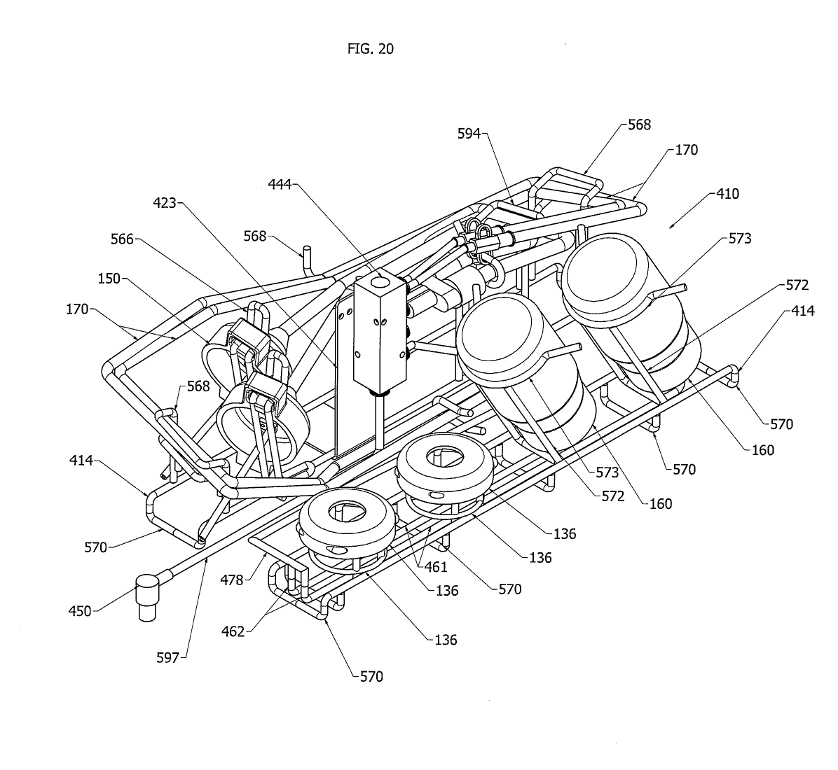

Yet another exemplary embodiment of a cleaning system 410 (e.g., a gastrointestinal (GI) endoscopic irrigation cleaning system), such as shown more generally in FIG. 1, shall be described with reference to FIGS. 19-27. FIG. 19 shows an exploded perspective view of the cleaning system 410 including the endoscopic components to be cleaned. FIG. 20 shows a perspective view of the cleaning system 410 with endoscopic components (e.g., water bottles 160, water bottle caps 150, and irrigation tubes 170, as well as bottle bases and O-rings 136) positioned in a non-walled container 412 (e.g., a non-walled container such as a rack structure including a plurality of holding structures) to be cleaned and a manifold apparatus 420. Unlike the embodiment described with reference to walled containers, non-walled container 412 (e.g., an open rack structure including dedicated structure corresponding to, and for receiving and maintaining the position of, components to be cleaned (e.g., the non-walled holding or rack apparatus is configured to clean multiple water bottle sets).

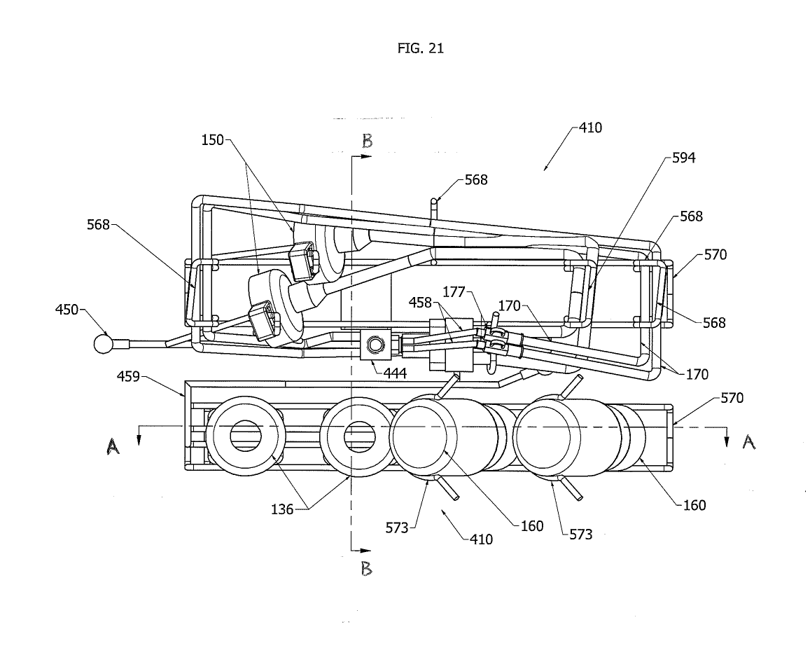

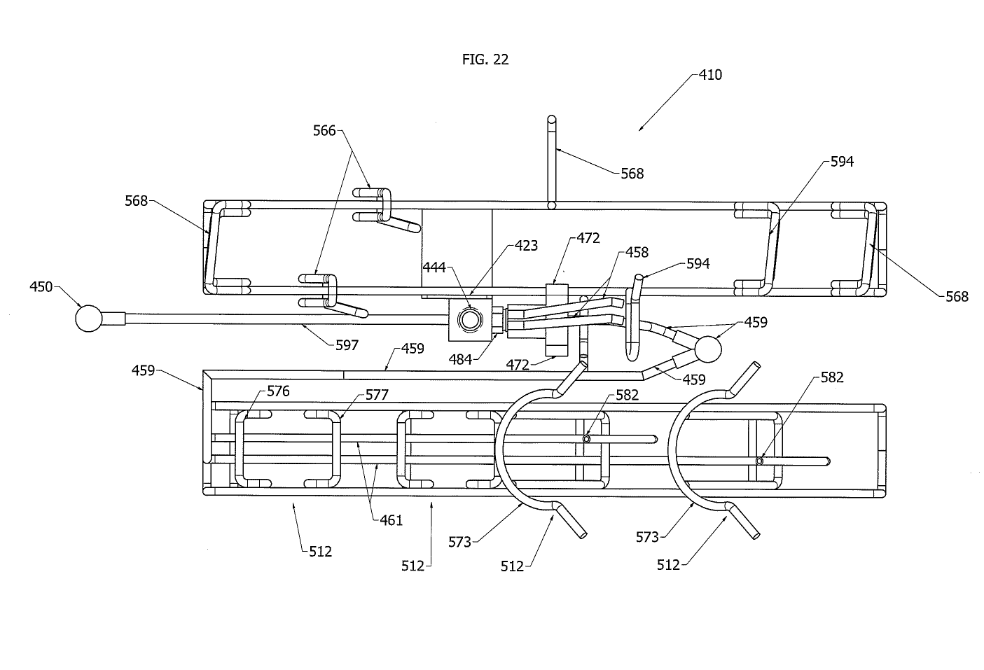

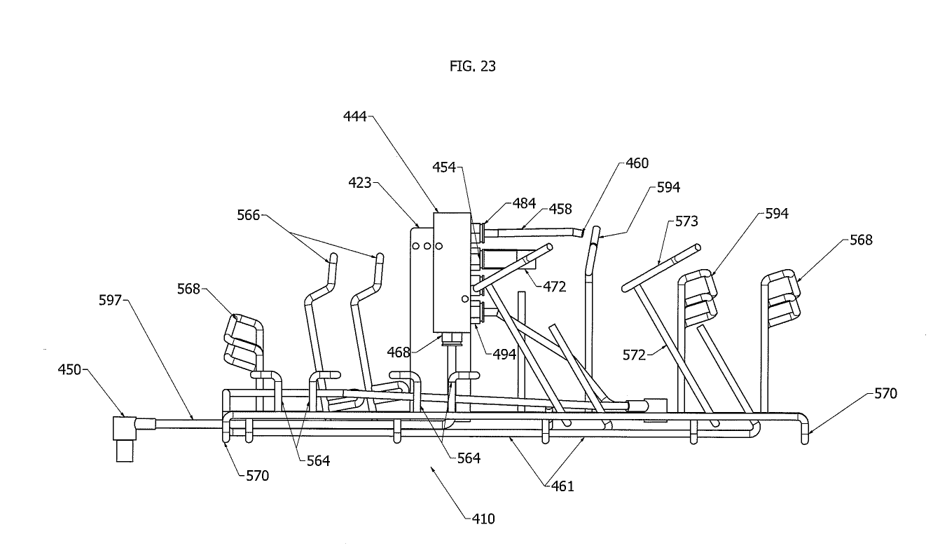

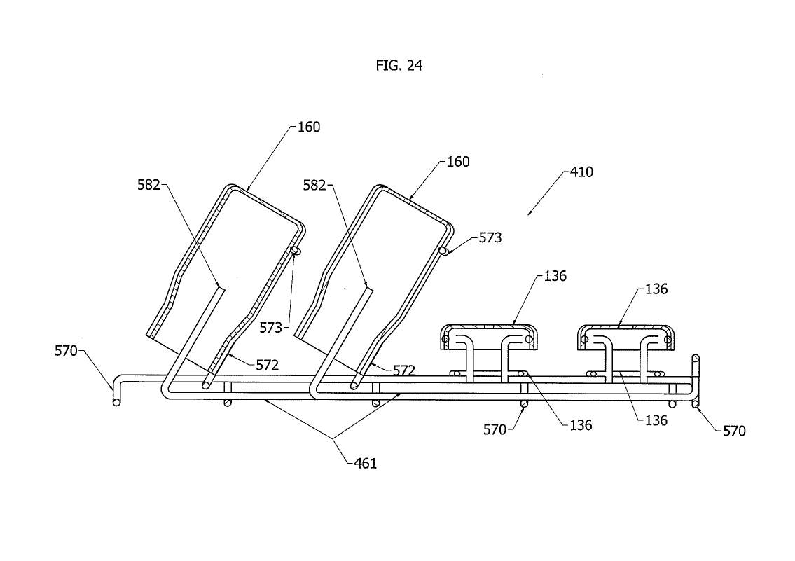

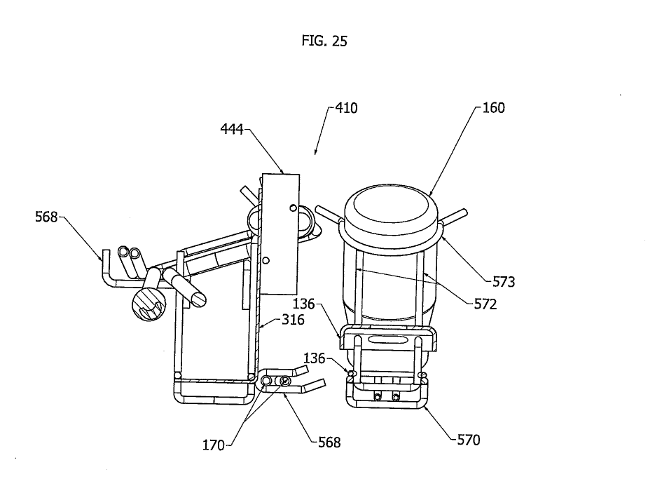

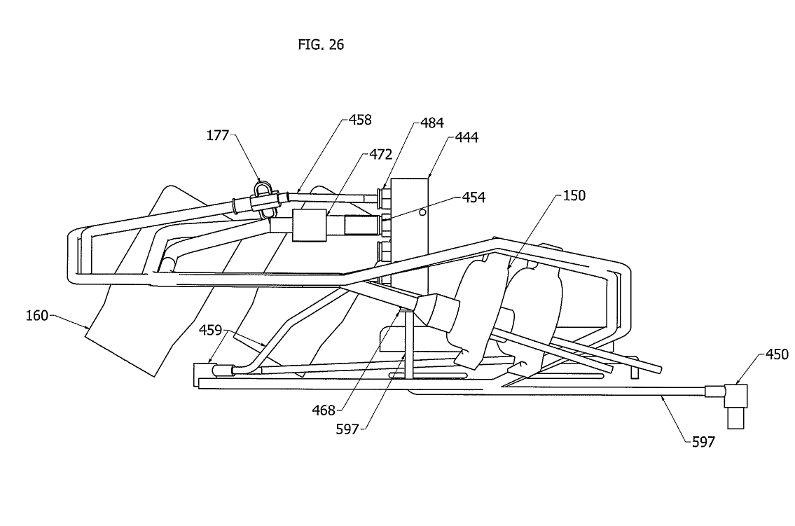

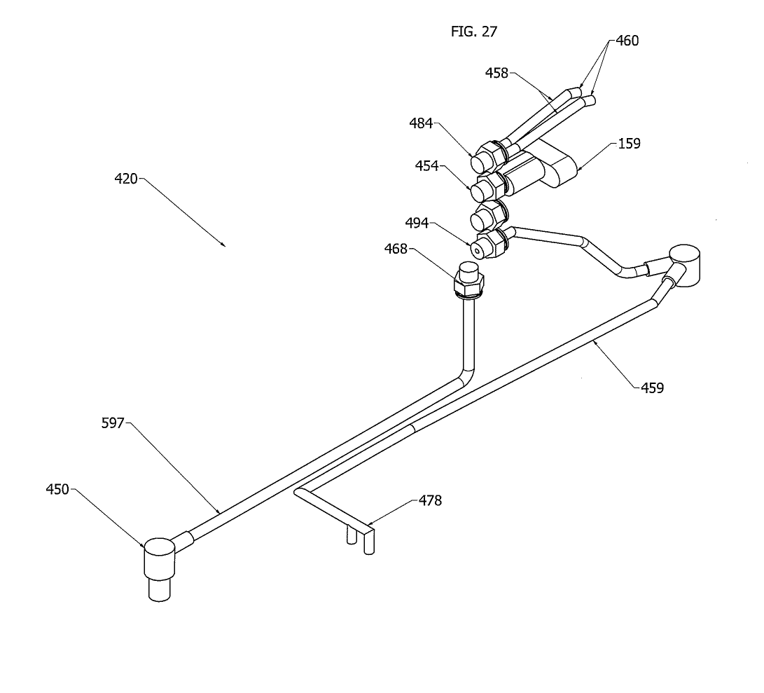

FIG. 21 shows a top view of the cleaning system 410 including the endoscopic components to be cleaned, while FIGS. 22 and 23 show a top view and a side view, respectively, of the cleaning system 410 with endoscopic components therein removed. FIG. 24 shows a cross-section view of at least a portion of the cleaning system 410 taken along line A-A as shown in FIG. 21 and FIG. 25 shows a cross-section view of the cleaning system 410 taken along line B-B also as shown in FIG. 21. Still further, FIG. 26 shows a side view of at least a portion of the exemplary manifold apparatus 420 (e.g., an assembly including various tubes, distribution elements, fittings, and connectors) with endoscopic components connected for cleaning and FIG. 27 shows a side perspective view of at least a portion of the exemplary manifold apparatus 420 used in the cleaning system 410 with endoscopic components removed.

The non-walled container 412 (e.g., a component containing or holding structure) of the cleaning system 410 as shown in FIGS. 19-27 includes a plurality of holding structures 512 (e.g., including retaining structures such as retaining structures 562 for assisting in holding water bottles 160 in position, retaining structures 564 for assisting in holding bottle bases and O-rings 136 in position, retaining structures 566 for assisting in holding water bottle caps 150 in position, and retaining structures 568 for assisting in holding irrigation tubes 170 in position). The holding structures 512 may be interconnected (e.g., with additional tubing or wire, or any other suitable structure) to form an integral unit or one or more portions of the non-walled container 412 that may or may not be interconnected (e.g., the portions may be coupled together in any manner, the manifold apparatus may be used to couple the portions, they may remain unconnected and positioned separately in the reprocessor chamber, etc.). As shown in FIGS. 19-27, the holding structures 512 may form two parts of the non-walled container (e.g., two holding or rack portions) that are coupled together using the manifold apparatus 420. At least in one embodiment, the materials used to form the non-walled container 412, provide a rigidity to make the container 412 non-collapsible (e.g., self-supporting or free-standing). Further, for example, the non-walled container 412 may include a bottom structure or structures 414 over which the various holding structures 512 are provided (e.g., connected, formed, coupled to, etc.). The bottom structure 414 may include any framework of elements (e.g., being open, or including one or more openings, to allow fluid flow therethrough). The openness of the non-walled container 412 (e.g., the framework of elements forming the bottom and the holding structures) allows fluids to contact the components effectively for cleaning as well as drain by gravity therefrom.

In one or more embodiments, the non-walled container 412 may be formed of a wire or tubing structure (e.g., or any other structural components) configured for receiving the components for cleaning. For example, the material forming the non-walled container (e.g., including the holding structures 512) may include any suitable type of material that provides one or more of the functions described herein (e.g., retaining the components, providing support for the components, allows fluid to reach the components and drain therefrom, etc.) including, but not limited to, wire, structure formed of polymers, metals, plastic, molded plastic, etc. Further, the container 412 may be formed of any suitable materials including, for example, stainless steel, plastic, molded plastic, noncorrosive metals, etc.

The non-walled container 412 (e.g., holding or rack apparatus) is sized, at least in one embodiment, to be positioned within a reprocessing chamber of a reprocessing apparatus (e.g., the basin of an AER apparatus). The container 412 may be of any size or shape, such as, but clearly not limited to, a rectangular or square bottom, or any other polygon shape.

In one or more embodiments, the bottom 414 of the container 412 is provided with standoff portions 570 so as to allow the components received to be positioned at a distance (e.g., in a non-contacting relationship) from a structure upon which it is placed. For example, when positioned within the basin of an AER apparatus, the bottom of the basin may be non-planar or include components mounted thereon. By providing standoffs 570, the container 412 may be positionable in any number of reprocessing chambers where such reprocessing chambers have different types of structures upon which the container 412 is to be placed. Further, such standoffs 570 may assist in providing space between the bottom of the basin of an AER apparatus and the components received by the holding structures 512.

In one or more embodiments, the container 412 may be provided as a single structure or as multiple structures coupled or uncoupled to one another. For example, the container 412 may be provided as two separate rack or holding structures each capable of holding a different water bottle set or any other components as desired. Further, for example, the container 412 may include various structural elements separating one or more portions thereof from other portions. For example, portions of the container 412 may be provided to hold certain of the one or more components. For example, one portion of the container may hold the water bottles while the other may hold the water bottle caps.

As shown in FIGS. 19-27, the plurality of holding structures 512 may correspond to and/or be configured to retain the various components being received thereby. For example, one or more holding structures 512 may include retaining structure 562 for assisting in holding water bottles 160 in position. For example, such retaining structure 562 includes a first portion 572 along which the water bottle 160 lies (e.g., the bottle 160 being held in an angular position relative to vertical and inverted such that water drains therefrom and off the bottom of the water bottle 160) and a second portion 573 (e.g., a curved portion) that is shaped to engage the cylindrical water bottle surface. In one embodiment, the curved portion 573 is sized to engage the water bottle surface and retain it in position as it is being cleaned (e.g., the second portion may be moved from a normal state (smaller than the diameter of the water bottle) to a larger engaging state when the bottle 160 is positioned therein, the arc of the curve may be more than 180 degrees to provide retention about the water bottle surface, etc.). The water bottle 160 is inverted and positioned over an outlet portion 582 through which fluid is provided into the interior of the bottle 160 when being cleaned.

Further, for example, one or more holding structures 512 may include retaining structure 564 for assisting in holding bottle bases and O-rings 136 in position. For example, such retaining structure 564 includes first and second portions 576, 577 which are insertable into a portion of the bottle base and O-ring 136 such that the bottle base is positioned over and held by the retaining structure 564 (e.g., the bottle base being held in an inverted position such that water drains therefrom and the O-ring being positioned or slipped over the first and second portions 576, 577 beneath the bottle base and held in place by the retaining structure and/or the bottle base). As shown, each of the first and second portions 576, 577 include opposing and outwardly extending members 578 for engaging the base 136 as, for example, shown in the cross-section of FIG. 24. Such outwardly extending members 578 may also be flexed inward to allow them to be inserted into the base 136 (e.g., to allow the base 136 to be place thereon) and then released so that they engage one or more inner surfaces of the base 136.

Still further, for example, one or more holding structures 512 may include retaining structure 566 for assisting in holding water bottle caps 150 in position. For example, such retaining structure 566 may terminate at a bent member 592 configured to be inserted into an opening in the cap closure portion 152 of the water bottle cap 150 (e.g., the cap 150 may hang on the bent member 592 such that the majority of the cap closure portion 152 is under the bent member 592 and is restricted from substantial movement). Further, retaining structure 594 may be formed to retain tube portions 154, 156 of the water bottle cap 150 when connected to the manifold apparatus 420 as described herein (e.g., retained in a position that allows the tube portions 154, 156 to gravity drain).

Yet still further, for example, one or more holding structures 512 may include retaining structure 568 for assisting in holding irrigation tubes 170 in position. For example, such retaining structure 568 may include spaced apart elements into which the irrigation tube 170 may be inserted and/or on which the tube 170 may lay. The retaining structure 568 is located such that the irrigation tubes 170 are positioned to allow them to gravity drain (e.g., one end being held higher than the other). It will be recognized that any type of retaining structure may be used to hold the various components. The present invention contemplates may other types of retaining structures to be suitable for holding the components in addition to those shown and described herein.

The manifold apparatus 420 of the cleaning system 410 as shown in FIGS. 9-27, at least in one embodiment, may be coupled to the non-walled container 412 by one or more attachment or coupling mechanisms (or such apparatus may be uncoupled or unconnected). For example, in one embodiment, an attachment panel 423 may be provided as part of the container 412 to which a fluid distribution element 444 of the manifold apparatus 420 may be attached using any suitable fastening or coupling technique, (e.g., adhesive, screw or bolt fasteners, hook and loop fasteners, etc.). The manifold apparatus 420 when coupled to the container 412 (e.g., a non-walled container including a plurality of holding structures) may assist to hold the components in specific locations (e.g., the outlets thereof being connectable to one or more portions of the components to be cleaned). Any suitable techniques may be used to effectively position the manifold apparatus 420, or any portions thereof, such that one or more components positioned relative to the container 412 can be connected as desired to the outlets of the manifold apparatus 420.

As shown in FIGS. 19-27, the exemplary manifold apparatus 420 may include any number of inlet connections, outlets, outlet connections, distribution elements, and/or tube portions suitable for carrying out the function of providing one or more fluids (e.g., cleaning solutions, disinfecting solutions, rinsing solutions, etc.) from the reprocessing apparatus to the one or more components being cleaned by the cleaning system 410 (e.g., a water bottle, a water bottle cap, irrigation tube, base holders, O-rings, etc.). For example, as described with reference to manifold apparatus 420 shown therein, an inlet connection 450 is provided for connection to a reprocessor fitting of a reprocessing apparatus to receive at least one fluid therefrom. The inlet connection 450 may be a connection compatible with the reprocessor fitting to provide a fluid tight connection therebetween. For example, the reprocessor fitting of a reprocessing apparatus is generally available within the reprocessing chamber for connection to, for example, an endoscope to be cleaned by the reprocessing apparatus. As such, the inlet connection 450 may be compatible with such a reprocessor fitting (e.g., the inlet connection may be a component from the reprocessor manufacturer, used to connect an endoscope, but now used as part of the manifold apparatus 420). Further, for example, the inlet connection 450 may include tubing for providing the connection to the reprocessor fitting.

In the embodiment shown in FIGS. 19-27, the inlet connection 450 (e.g., including a reprocessor manufacturer's part compatible with the reprocessor fitting of a specific AER) is connected by tubing 597 to a connection fitting 468 (see FIGS. 23 and 27). The connection fitting 468 is configured to mate with an inlet port 481 of distribution element 444 (see FIG. 23). However, the inlet connection 450 may be connected to the distribution element 444 in any manner or configuration. The distribution element 444 may include any number of inlet and outlet ports and be provided by any configuration of elements and tubing (e.g., a multiple port apparatus, one or more tee fittings connected together, etc.).

The distribution element 444, for example, may provide fluid from the inlet connection 450 to an outlet port 484 thereof. The outlet port 484 may be fluidly connected to bifurcated tube channels 458 each terminating at an outlet connection 460. Each of the outlet connections 460, for example, may be compatible for connection to the tube fitting 177 of an irrigation tube 170 for use in flushing fluid through the irrigation tube 170 during the cleaning cycle. The irrigation tubes 170 may be held at various levels of elevation by the retaining structures 568 (e.g., which may form the irrigation tube into a gradually descending loop when positioned therein) or any other suitable structure such as shown in, for example, FIG. 20 so as to position the irrigation tubes 170 for gravity draining.

The distribution element 444, for example, may also provide fluid from the inlet connection 450 to an outlet port 454. The outlet port 454 may be fluidly connected to an outlet connection 472 (e.g., a tee connector). The outlet connections 472, for example, may be compatible for connection to the tube fittings 159 of water bottle caps 150 for use in flushing fluid through the tubes 154, 156 of the water bottle cap 150. The tubes 154, 156 may be held at various levels of elevation by retaining structures 594 or any other suitable structure such as shown in, for example, FIG. 20 so as to position the tubes 154, 156 for gravity draining.

Still further, the distribution element 444, for example, may provide fluid from the inlet connection 450 to an outlet port 494. The outlet port 494 may be fluidly connected to a tubing 459 by one or more elements, which then bifurcates and terminates at two tube ends 478. Each of the two tube ends 478 are shaped and/or configured to provide a structure for connection to an additional multiple line fluid transfer outlet element 461 (e.g., which may be attached to a portion of the container 421, may be part of a holding structure for receiving a water bottle 160, etc.). The multiple line fluid transfer outlet element 461 includes a first end including two inlets 462 which may be coupled to the two tube ends 478 and tubing which extends from the first end terminating at distal tube portions 582 upon which a water bottle 160 may be positioned (e.g., structure suitable to receive a water bottle in an inverted position allowing fluid to be provided into the interior of the water bottle 160 and that allow the bottles to drain by gravity, portions that may include a spray element, portions that are like those described with reference to manifold 320, etc.).

Although particular configurations of manifold apparatus are described in detail herein with respect to both walled and non-walled containers, one skilled in the art will recognize that any number of different components (e.g., flexible or inflexible), such as tubes, connections, distribution elements, tee connectors, metal or plastic parts, etc., connectable in any manner, may be utilized to provide the manifold apparatus. Further, for example, the manifold apparatus may be formed of any number of different materials such as, but clearly not limited to plastics, stainless steel, other suitable metals or metal alloys or one or more polymers, etc.

The various outlet connections of the manifold apparatus described herein with respect to both walled and non-walled containers, at least in one or more embodiments, may be compatible with the fittings of the various components to be cleaned by the cleaning systems. However, one will recognize that adapters may also be used for adapting an outlet connection to be compatible with such fittings. Such adapters may also be applicable in the fluid connection between the inlet connection and the reprocessor fitting. As used herein, the term connection, connector, fitting, or the like encompasses the use of an adapter.

In one or more embodiments, the cleaning system (e.g., which could be referred to as the Endo 360) gives a user confidence in the ability to use re-usable products in a cost effective method driven by using good sound repeatable cleaning practices (e.g., replication of the cleaning process which meets the standards of the manufacturer). Conventionally, a very low standard of cleaning repeatability exists for the support team that has to clean and sign off on such components being cleaned. The manufacturer's guidelines are cumbersome, which has led to the use of disposable water bottles and tube sets that are disposed of daily. In some instances, they are disposed of after each patient use. The cleaning system was developed to be able to utilize a method of cleaning that is accepted in the AER of the user's choice. The cleaning system may use a currently used AER and is configured such that all of the re-usable water bottles and tube sets, as well as accessories, may be positioned in a defined pattern which is easily repeatable. Further, the cleaning system may only allow a certain method to put each re-usable component in its particular and certain place or location. This reduces that chance of mistakes, provides great accountability, and provides a standardized method that allows repeatable and acceptable cleaning practices.

For example, in one embodiment, the dirty water bottle may be taken apart, resulting in a water bottle, an O-ring, a water bottle base, and water bottle cap all apart. Each component may be put into the designated assigned locations in the cleaning system (e.g., the walled container such as a basket or the non-walled container such as the rack structure including a plurality of holding structures for the components). The water bottle will be connected or positioned on the tube portion of the manifold apparatus in its designated position. There may be a clamp or other structure to help hold the proximal end of the tube of the water bottle cap in its correct place. Such components may be positioned in their location in approximately one minute or less. Repeatability is easy for training and cross training team members, as the products may be positioned in the system only in one way.

The irrigation tube may be put in last. The irrigation tube may be hooked up to it at an assigned spot or location. For example in the walled container, it may be wrapped around on either the inside of the basket or container of cleaning system or the outside of the basket or container (e.g., cage) depending on the AER or cleaning system being used. The position of each component may allow for gravity to play a part in helping drain any leftover cleaning fluid therein. Most AER's use an alcohol or air blown through the channels to help clear the tube sets, but one or more of the components (and even all parts) may be positioned to use gravity to help. Simplicity and repeatability may be the mainstay of the cleaning system. There are no specific and consistent clear methods to reprocessing manufacturer's water bottles and tube sets. One or more of the cleaning systems herein will be the first product usable with any of the manufacturer's water bottles and tube sets to completely change the disposable methods currently being used, and make use of quality re-usable products (e.g., decreasing the amount of disposable product sent to landfills in support of the current green movement of disposing of unnecessary disposable products). One or more of the cleaning systems described herein will be the only system choice to accomplish such functions and standardize the cleaning of irrigation bottles and tube sets.

All patents, patent documents, and references cited herein are incorporated in their entirety as if each were incorporated separately. This disclosure has been provided with reference to illustrative embodiments and is not meant to be construed in a limiting sense. As described previously, one skilled in the art will recognize that other various illustrative applications may use the techniques as described herein to take advantage of the beneficial characteristics of the apparatus and methods described herein. Various modifications of the illustrative embodiments, as well as additional embodiments of the disclosure, will be apparent upon reference to this description.

What is claimed is:

1. A cleaning system for use with an automated endoscopic reprocessing apparatus for cleaning one or more components used in an endoscopic medical procedure, wherein the automated endoscopic reprocessing apparatus comprises a reprocessor chamber and a reprocessor fitting configured to provide at least one fluid, wherein the at least one fluid comprises at least one of a cleaning solution, a disinfecting solution, and a rinsing solution, and further wherein the one or more components comprise at least one of one or more water bottles, one or more water bottle caps, and one or more irrigation tubes used in an endoscopic procedure, wherein each water bottle cap comprises at least one tube therethrough terminating at a tube fitting connectable to an endoscopic system used in an endoscopic procedure, wherein each irrigation tube extends from a first end to a second end with at least one of the first and second ends terminating at a tube fitting connectable to an endoscopic system used in an endoscopic procedure, the cleaning system comprising:
a container to receive and hold the one or more components; and
a manifold apparatus, wherein the manifold apparatus comprises:
an inlet connection connectable to the reprocessor fitting of the automated endoscopic reprocessing apparatus to receive the at least one fluid therefrom, and
one or more outlets configured to provide the at least one fluid into or through the one or more components, wherein the one or more outlets comprise at least one outlet connection connectable to at least one tube fitting of at least one water bottle cap or at least one irrigation tube.

2. The cleaning system of claim 1, wherein the container comprises a walled container comprising at least a bottom and one or more side walls, wherein at least portions of the bottom and one or more side walls comprise a network of material providing a plurality of openings to allow fluids to pass therethrough to contact the one or more components.

3. The cleaning system of claim 1, wherein the container comprises a non-walled container comprising a plurality of holding structures, wherein each of the plurality of holding structures is configured to receive at least one component of the one or more components such that fluid is allowed to contact the one or more components.

4. The cleaning system of claim 3, wherein the non-walled container is configured as at least one rack apparatus including a bottom structure relative to which one or more of the plurality of holding structures are arranged to receive the one or more components.

5. The cleaning system of claim 1, wherein the manifold apparatus further comprises one or more manifold tube portions fluidly connected in the path from the inlet connection to the one or more outlets, wherein at least one of the one or more manifold tube portions comprises an outlet terminating a tube portion extending in a direction such that an open end of the water bottle is positionable onto the tube portion.

6. The cleaning system of claim 1, wherein the one or more components comprise at least one water bottle, at least one water bottle cap, and at least one irrigation tube used in an endoscopic procedure, wherein the one or more outlets comprise at least one outlet connection connectable to a tube fitting of the at least one water cap bottle and at least one outlet connection connectable to a tube fitting of the at least one irrigation tube, wherein the manifold apparatus further comprises one or more manifold tube portions fluidly connected in the path from the inlet connection to the one or more outlets, wherein at least one of the one or more manifold tube portions comprises an outlet terminating a tube portion extending in a direction such that an open end of the at least one water bottle is positionable onto the tube portion.

7. The cleaning system of claim 1, wherein the container is sized to be received in the reprocessing chamber of the automated endoscopic reprocessing apparatus.

8. A method of cleaning one or more components used in a medical procedure, the cleaning method comprising:
providing a reprocessing apparatus comprising a reprocessing chamber and a reprocessor fitting through which at least one fluid is provided, wherein the at least one fluid comprises at least one of a cleaning solution, a disinfecting solution, and a rinsing solution; and
providing a cleaning system comprising:
a container to receive and hold the one or more components; and
a manifold apparatus, wherein the manifold apparatus comprises:
an inlet connection connectable to the reprocessor fitting of the reprocessing apparatus to receive the at least one fluid therefrom, and
one or more outlets configured to provide the at least one fluid into or through the one or more components, wherein the one or more outlets comprise at least one outlet connection connectable to a component fitting of at least one of the one or more components;
receiving at least one of the one or more components by the container, wherein receiving that at least one component comprises connecting the at least one outlet connection to the component fitting of the at least one component;
positioning the container into the reprocessing chamber, wherein positioning the container into the reprocessing chamber comprises connecting the inlet connection to the reprocessor fitting of the reprocessing apparatus; and
providing the at least one fluid into or through at least one of the one or more components positioned into the reprocessing chamber.

9. The method of claim 8, wherein the one or more components comprise at least one of a water bottle, a water bottle cap, and an irrigation tube used in an endoscopic procedure, and further wherein the inlet connection of the manifold apparatus is connectable to a reprocessor fitting of an automated endoscopic reprocessing apparatus from which it receives the at least one fluid.

10. The method of claim 8, wherein the container comprises a walled container comprising at least a bottom and one or more side walls, wherein at least portions of the bottom and one or more side walls comprise a network of material providing a plurality of openings to allow fluids to pass therethrough to contact the one or more components.

11. The method of claim 8, wherein the container comprises a non-walled container comprising a plurality of holding structures, wherein each of the plurality of holding structures is configured to receive at least one component of the one or more components such that fluid is allowed to contact the one or more components.

12. The method of claim 11, wherein the non-walled container is configured as at least one rack apparatus including a bottom structure relative to which one or more of the plurality of holding structures are arranged to receive the one or more components.

13. The method of claim 8, wherein the manifold apparatus further comprises one or more manifold tube portions fluidly connected in the path from the inlet connection to the one or more outlets, wherein at least one of the one or more manifold tube portions comprises an outlet insertable into an interior volume of at least one of the one or more components.

14. The method of claim 13, wherein the one or more components comprise at least one water bottle used in an endoscopic procedure, wherein the at least one manifold tube portion comprises an outlet terminating a tube portion extending in a direction such that an open end of the water bottle is positionable onto the tube portion.

15. The method of claim 8, wherein the one or more components comprise at least one of a water bottle cap used in an endoscopic procedure, the water bottle cap comprising at least one tube therethrough terminating at a tube fitting connectable to an endoscopic system used in an endoscopic procedure, and further wherein the at least one outlet connection of the one or more outlets of the manifold apparatus is connectable to the tube fitting of the at least one tube extending through the water bottle cap.

16. The method of claim 8, wherein the one or more components comprise at least one irrigation tube used in an endoscopic procedure, wherein the irrigation tube extends from a first end to a second end with at least one of the first and second ends terminating at a tube fitting connectable to an endoscopic system used in an endoscopic procedure, and further wherein the at least one outlet connection of the one or more outlets of the manifold apparatus is connectable to the tube fitting of the at least one irrigation tube.

17. A cleaning system for use with a reprocessing apparatus for cleaning one or more components used in a medical procedure, the cleaning system comprising:
at least one rack apparatus including a bottom structure relative to which one or more of a plurality of holding structures are arranged to receive the one or more components; and
a manifold apparatus, wherein the manifold apparatus comprises:
an inlet connection connectable to a reprocessor fitting of the reprocessing apparatus to receive at least one fluid therefrom, wherein the at least one fluid comprises at least one of a cleaning solution, a disinfecting solution, and a rinsing solution, and
one or more outlets configured to provide the at least one fluid into or through one or more components received by the at least one rack apparatus, wherein the one or more outlets comprise at least one outlet connection connectable to a component fitting of at least one of the one or more components.

18. The cleaning system of claim 17, wherein the one or more components comprise at least one of a water bottle, a water bottle cap, and an irrigation tube used in an endoscopic procedure, and further wherein the inlet connection of the manifold apparatus is connectable to a reprocessor fitting of an automated endoscopic reprocessing apparatus from which it receives the at least one fluid.

19. The cleaning system of claim 17, wherein the manifold apparatus further comprises one or more manifold tube portions fluidly connected in the path from the inlet connection to the one or more outlets, wherein at least one of the one or more manifold tube portions comprises an outlet insertable into an interior volume of at least one of the one or more components.

20. The cleaning system of claim 19, wherein the one or more components comprise at least one water bottle used in an endoscopic procedure, wherein the at least one manifold tube portion comprises an outlet terminating a tube portion extending in a direction such that an open end of the water bottle is positionable onto the tube portion.

21. The cleaning system of claim 17, wherein the one or more components comprise at least one of a water bottle cap used in an endoscopic procedure, the water bottle cap comprising at least one tube therethrough terminating at a tube fitting connectable to an endoscopic system used in an endoscopic procedure, and further wherein the at least one outlet connection of the one or more outlets of the manifold apparatus is connectable to the tube fitting of the at least one tube extending through the water bottle cap.

22. The cleaning system of claim 17, wherein the one or more components comprise at least one irrigation tube used in an endoscopic procedure, wherein the irrigation tube extends from a first end to a second end with at least one of the first and second ends terminating at a tube fitting connectable to an endoscopic system used in an endoscopic procedure, and further wherein the at least one outlet connection of the one or more outlets of the manifold apparatus is connectable to the tube fitting of the at least one irrigation tube.

23. The cleaning system of claim 22, wherein at least one holding structure of the plurality of holding structures comprises one or more retaining elements used to secure the at least one irrigation tube in a manner allowing the at least one irrigation tube to gravity drain.

24. The cleaning system of claim 17, wherein at least one holding structure of the plurality of holding structures comprises at least one of one or more retaining elements insertable into at least a portion of at least one component of the one or more components to maintain the at least one component in a position when fluid is allowed to contact the at least one component, one or more retaining elements that engage at least a surface portion of at least one component of the one or more components to maintain the at least one component in a position when fluid is allowed to contact the at least one component, and one or more retaining elements into which at least a portion of at least one component of the one or more components is insertable to maintain the at least one component in a position when fluid is allowed to contact the at least one component.

25. The cleaning system of claim 17, wherein each of the plurality of holding structures is configured to receive at least one component of the one or more components and maintain the at least one component in a position when fluid is allowed to contact the one or more components.

26. The cleaning system of claim 17, wherein the manifold apparatus is coupled to the at least one rack apparatus.
